(19) United States
(12) Patent Application Publication
(10) Pub. No.: US 2014/0303719 A1
(43) Pub. Date: Oct. 9, 2014

(54) PERCUTANEOUSLY IMPLANTABLE ARTIFICIAL HEART VALVE SYSTEM AND ASSOCIATED METHODS AND DEVICES

(71) Applicants: Brian J. Cox, Laguna Niguel, CA (US); Robert Rosenbluth, Laguna Niguel, CA (US); Paul Lubock, Monarch Beach, CA (US)

(72) Inventors: Brian J. Cox, Laguna Niguel, CA (US); Robert Rosenbluth, Laguna Niguel, CA (US); Paul Lubock, Monarch Beach, CA (US)

(73) Assignee: INCEPTUS MEDICAL, LLC, Aliso Viejo, CA (US)

(21) Appl. No.: 14/232,238

(22) PCT Filed: Jun. 22, 2012
(86) PCT No.: PCT/US12/43885

§ 371 Date: Jun. 17, 2014

Related U.S. Application Data
(60) Provisional application No. 61/501,148, filed on Jun. 24, 2011.
(60) Provisional application No. 61/508,015, filed on Jul. 14, 2011.
(60) Provisional application No. 61/583,993, filed on Jan. 6, 2012.

(51) Int. Cl. A61F 2/24

(52) U.S. Cl. CPC A61F 2/2445; A61F 2/2466
USPC 623/2.11; 623/2.37

(57) ABSTRACT

Expandable prosthetic valve devices for repair or replacement of a native valve in a heart of a patient and associated, systems and methods are disclosed herein. An expandable prosthetic valve device configured in accordance with a particular embodiment of the present technology can include a radially-expandable support having an expandable outer wall and a lumen defined by the outer wall. The device can also include a valve in the lumen and coupled to the support and a self-expanding retainer coupled to the outer wall. The retainer can have a structural braid configured to form a first annular flange on the outer wall of the support, and an occlusive braid configured to reduce blood flow through the retainer.

### RELATED APPLICATIONS INCORPORATED BY REFERENCE

The present application claims priority to U.S. Provisional Patent Application No. 61/501,148, filed Jun. 24 2011, entitled “PERCUTANEOUSLY IMPLANTABLE ARTIFICIAL HEART VALVE SYSTEM AND METHOD,” to U.S. Provisional Patent Application No. 61/508,015, filed Jul. 14, 2011, entitled “PERCUTANEOUSLY IMPLANTABLE ARTIFICIAL HEART VALVE SYSTEM AND METHOD,” and to U.S. Provisional Patent Application No. 61/583,993, filed Jan. 6, 2012, entitled “DEVICES AND METHOD FOR OCCLUSION OF THE LEFT ATRIAL APPENDAGE,” all of which are incorporated herein in their entireties by reference. As such, components and features of embodiments disclosed in the applications incorporated by reference may be combined with various components and features disclosed and claimed in the present application.

### TECHNICAL FIELD

The present technology relates generally to artificial replacement heart valves and associated systems and methods. In particular, several embodiments are directed to expandable prosthetic heart valve devices and methods for minimally invasive implantation, such as percutaneous implantation, of expandable prosthetic heart valve devices.

### BACKGROUND

The human heart is a muscular organ that provides continuous blood circulation through the cardiac cycle. The heart can be divided into four main chambers called the right and left atria and the right and left ventricles. The right heart, containing the right atrium and ventricle, and are separated by a muscular wall or septum from the left heart, containing the left atria and ventricle. The right heart supplies the lung (pulmonary) circulation while the left heart supplies the remaining circulation to the body. To insure that blood flows in one direction from the right to the left heart, atrioventricular valves are present at the inlet junctions of the atria and the ventricles (the tricuspid valve on the right and the mitral valve on the left), and semi-lunar valves (the pulmonary valve on the right and the aortic valve on the left) govern the exits of the ventricles leading to the lungs and the rest of the body. These valves contain leaflets that open and shut in response to blood pressure changes caused by the contraction and relaxation of the heart chambers.

Diseases of the heart valves are common and can include valvular stenosis, while the opening through the valve is smaller than normal causing the heart to work harder to pump, and valvular insufficiency or regurgitation, where the valve does not close completely, allowing blood to flow backwards and causing the heart to be less efficient. These diseases may be congenital or acquired through infections such as endocarditis or rheumatic fever as well as drug use or age related degeneration. Symptoms such as shortness of breath, weakness, dizziness, fainting, palpitations, anemia and edema may be present and are often severe enough to be debilitating and/or life threatening.

Surgically implantable artificial heart valves for replacing damaged or diseased native valves are commonly used in clinical practice today, particularly in the aortic and mitral positions. These replacement valves can be the “tissue” type—constructed with mammalian tissues on polymeric or metal supports, or the “mechanical” type where no tissue is used and the device is fabricated from biocompatible metals, ceramics and polymers. Current implantation procedures are performed under general anesthesia and typically require division of the rib cage at the sternum to access the heart and major blood vessels. Patients are placed on a cardiopulmonary bypass machine for several hours in which the heart is stopped and the replacement valve is positioned in the remnant valve annulus. An annular sewing or suture ring, often composed of a polymer fabric such as Dacron®, surrounds the valve frame to which the surgeon sutures the replacement valve to a remnant valve annulus. The latter task can take up to 45 to 90 minutes with a skilled cardiac surgeon. Consequently, many patients who are in need of a valve replacement are excluded due to the severity and risks associated with this highly invasive surgical procedure.

Specialized annulus attachment rings have been proposed as substitutes for commonly used fabric sewing rings in order to reduce operation times. Such rings could be attached. without suturing in a few minutes and are disclosed, for example, in U.S. Pat. Nos. 3,143,742 and 3,464,065 to Cromie, the contents of which are hereby incorporated by reference. Collapsible tissue valves incorporating an expandable stent framework have been proposed to eliminate or greatly reduce the time needed for suturing. Such expandable stents are disclosed, for example. in U.S. Pat. No. 3,657,744 to Ersek, the contents of which are hereby incorporated by reference. Advances in minimally invasive surgical and interventional cardiology techniques have led to valve replacements that are performed through intercostal, transseptal, transapical, transfemoral and other less invasive and percutaneous approaches in attempts to lessen the morbidity and mortality risks of these procedures.

These and other replacement heart valve systems have a number of potential drawbacks particularly When attempting to adapt them to the mitral position. The mitral valve is typically oval or kidney-shaped, unlike the circular or more uniform aortic valve, and includes clusters of chordae tendineae extending from the valve leaflets to the papillary muscles located at the posterior surface of the left ventricle. Moreover, the mitral valve annulus has muscle only along the outer wall of the valve and the thin vessel wall that separates the mitral valve and the aortic valve can cause distortion of the mitral valve annulus. Thus, conventional expandable stents, which are typically cylindrical in shape and apply only radial force against the annulus, are limited for treating conditions of the mitral valve.

For example, conventional stents, can cause insufficient sealing around the mitral valve annulus leading to paravalvular leaking (regurgitation) due to the high pressures experienced on left ventricular contraction. They may also suffer from inadequate fixation around the mitral annulus leading to valve dislodgement or improper placement due to the high pressure and anatomical challenges such as the presence of chordae tendineae and remnant leaflets, leading to valve impingement. Additional challenges are present for accurate valve positioning and seating during percutaneous delivery, collapsing and maintaining flexibility of the device during delivery in order to reliably navigate blood vessels and pass benignly through the aortic valve to the mitral position, and promoting natural tissue ingrowth and healing of the artificial annulus following implantation. Accordingly, there is a strong public-health need for alternative treatment strategies.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. Furthermore, components can be shown as transparent in certain views for clarity of illustration only and not to indicate that the illustrated component is necessarily transparent.

FIG. 1 is a schematic illustration of a cross-sectional view of a heart depicting the major chambers, blood vessels, blood flow patterns and anatomical features of the heart and showing an expandable prosthetic valve device implanted at the native mitral valve in accordance with an embodiment of the present technology.

FIG. 2A is a side view of an expandable prosthetic valve device for implantation at a native valve region of a heart shown in a deployed state (e.g., expanded configuration) and configured in accordance with an embodiment of the present technology.

FIGS. 2B-2C are perspective and top views, respectively, of the device as configured in FIG. 2A.

FIG. 3 is an isometric view of an expandable prosthetic valve device for implantation at a native valve region of a heart and in a deployed state (e.g., expanded configuration) configured in accordance with another embodiment of the present technology.

FIGS. 4A-4B are cross-sectional side views of portions of self-expanding braids configured in accordance with various embodiments of the present technology.

FIG. 5 is a side view of a mandrel and a braided mesh formed over the mandrel configured in accordance with an embodiment of the present technology.

FIG. 6 is an enlarged view of a self-expanding braid with interwoven large and small strands configured in accordance with an embodiment of the present technology.

FIG. 7 is an enlarged cross-sectional side view of select components of an expandable prosthetic valve device implanted at a native valve annulus in the heart in accordance with an embodiment of the present technology.

FIG. 8A is an enlarged cross-sectional view of an expandable prosthetic valve device shown in a deployed state (e.g., expanded, configuration) configured in accordance with an embodiment of the present technology.

FIG. 8B is an enlarged cross-sectional view the expandable prosthetic valve device of FIG. 8A shown in a delivery state (e.g., low-profile or collapsed configuration) configured in accordance with an embodiment of the present technology.

FIG. 8C is a side view of the expandable prosthetic valve device as configured in FIG. 8A configured in accordance with an embodiment of the present technology,

FIG. 8D is a side view the expandable prosthetic valve device as configured in FIG. 8C implanted at a native mitral valve in the heart in accordance with an embodiment of the present technology.

FIGS. 9A-9B are top views of an expandable prosthetic valve devices having a plurality of fixation members configured in accordance with embodiments of the present technology.

FIGS. 10A-10L are cross-sectional side views of portions of expandable prosthetic valve devices in delivery and deployed states configured in accordance with various embodiments of the present technology.

FIGS. 11A-11I are cross-sectional side views of portions of expandable prosthetic valve devices in delivery and deployed states configured in accordance with additional embodiments of the present technology.

FIG, 12A is a side view of an expandable prosthetic valve device showing a self-expanding braid transitioning from a delivery state to a deployed state configured in accordance with an additional embodiment of the present technology.

FIGS. 12B-12C are enlarged cross--sectional side views of expandable prosthetic valve devices configured in accordance with embodiments of the present technology.

FIG. 12D is a cross-sectional side view the prosthetic valve device of FIG. 12C implanted at a native mitral valve in the heart in accordance with an embodiment of the present technology.

FIGS. 13B-13C are enlarged cross-sectional views of expandable prosthetic valve devices configured in accordance with additional embodiments of the present technology.

FIG. 14 illustrates a prosthetic valve device delivery system in accordance with an embodiment of the present technology.

FIG. 15 is a schematic illustration of a cross-sectional vim of a heart showing a guidewire traveling along a guidewire path through the heart in accordance with an embodiment of the present technology.

FIGS. 16A-16J are schematic illustrations of embodiments of attachment mechanisms suitable for coupling surgical components percutaneously within a target chamber of a heart configured in accordance with various embodiments of the present technology.

### DETAILED DESCRIPTION

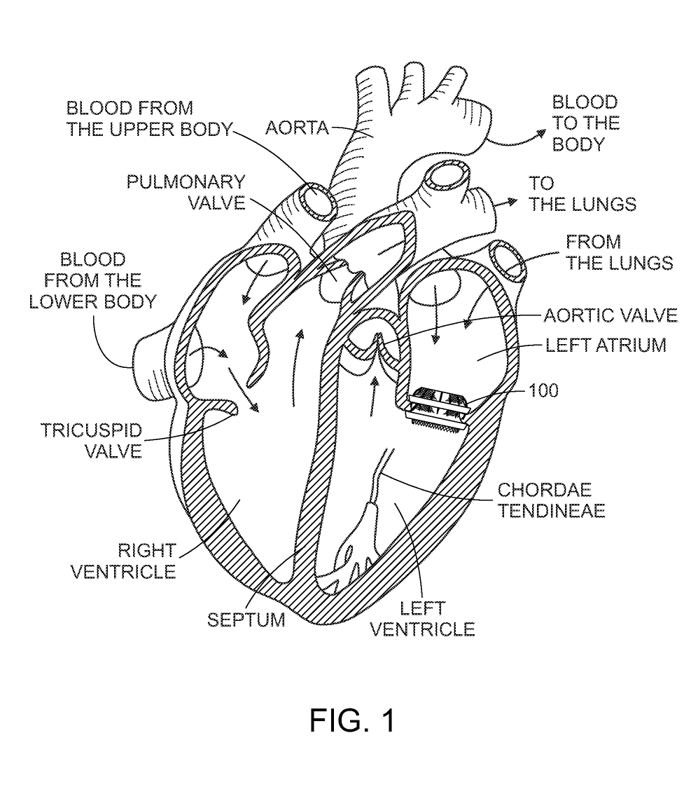

Specific details of several embodiments of the technology are described below with reference to FIGS. 1-16J. Although many of the embodiments are described below with respect to devices, systems, and methods for treatment of heart valve diseases and conditions by percutaneous implantation of expandable prosthetic valves, other applications and other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described below with reference to FIGS. 1-16J.

With regard to the terms “distal” and “proximal” within this description, unless otherwise specified, the terms can reference a relative position of the portions of a prosthetic valve device and/or an associated delivery device with reference to an operator and/or a location in the vasculature. For example, proximal can refer to a position closer to the operator of the device or an incision into the vasculature, and distal can refer to a position that is more distant from the operator of the device or further from the incision along the vasculature. With respect to a prosthetic heart valve device, the terms “proximal” and “distal” can refer to the location of portions of the device with respect to the direction of blood flow. For example, proximal can refer to an upstream position or a position of blood inflow, and distal can refer to a downstream position or a position of blood outflow. For ease of reference, throughout this disclosure identical reference numbers are used to identify similar or analogous components or features, but the use of the same reference number does not imply that the parts should be construed to be identical. Indeed, in many examples described herein, the identically numbered parts are distinct in structure and/or function. The headings provided herein are for convenience only.

### Selected Embodiments of Artificial Heart Valve Systems and Devices

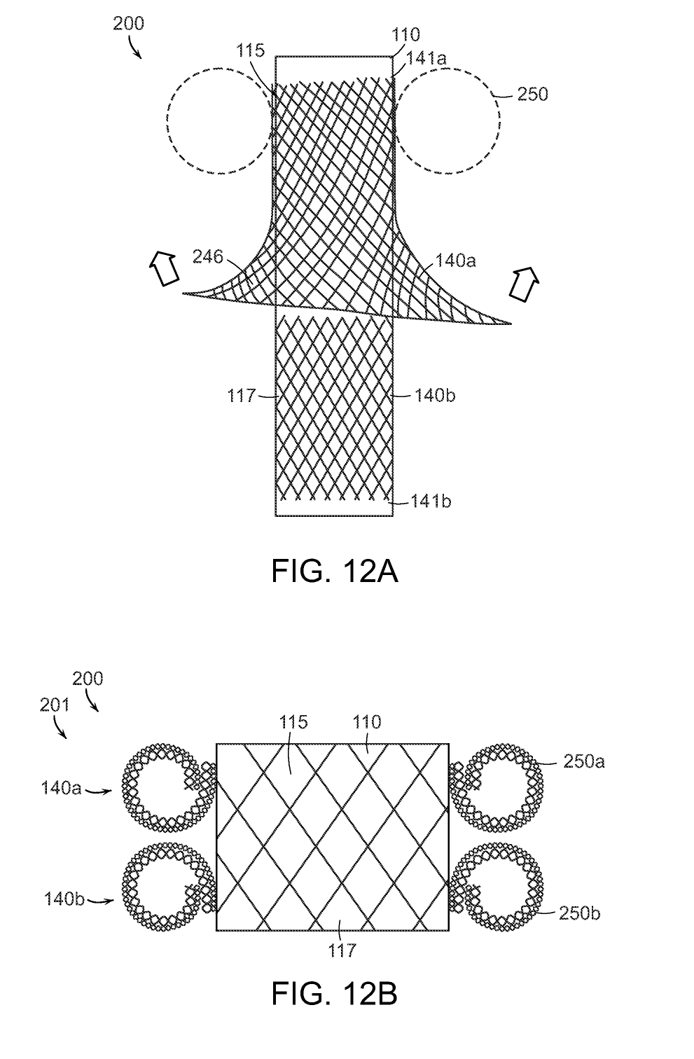

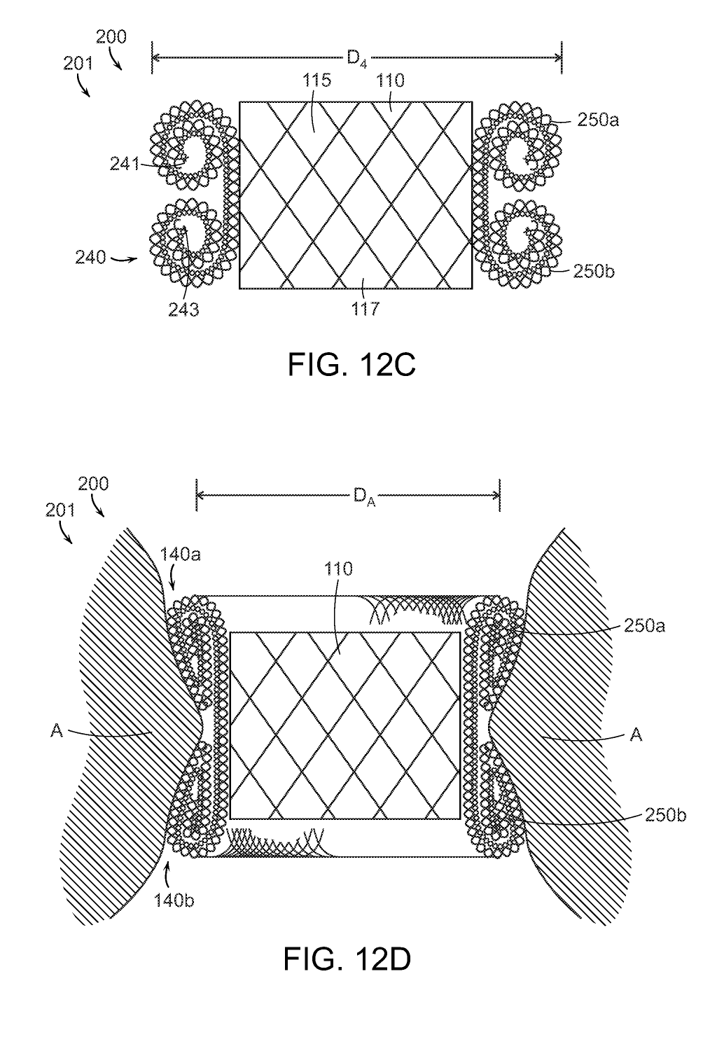

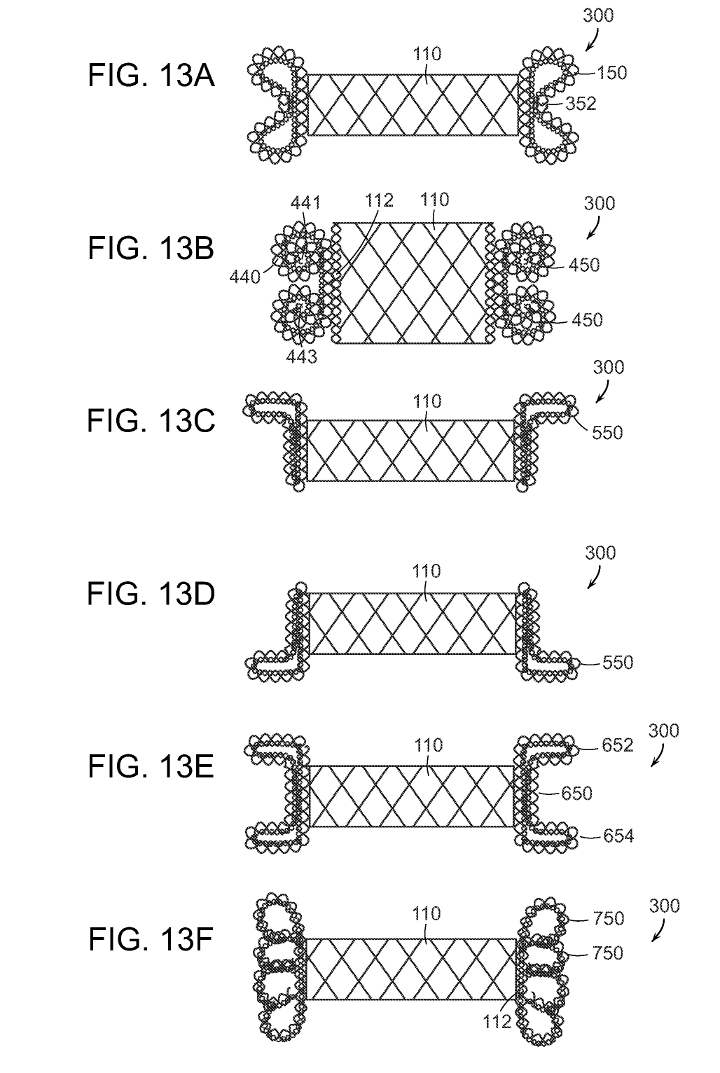

Introductory examples of artificial heart valve systems, system components and associated methods in accordance with embodiments of the present technology are described in this section with reference to FIGS. 1-11I. It will be appreciated that specific elements, substructures, advantages, uses, and/or other features of the embodiments described with reference to FIGS. 1-11I can be suitably interchanged, substituted or otherwise configured with one another and/or with the embodiments described with reference to FIGS. 12A-13F in accordance with additional embodiments of the present technology. Furthermore, suitable elements of the embodiments described with reference to FIGS. 1-13F can be used as stand-alone and/or self-contained devices.

Systems, devices and methods are provided herein for percutaneous implantation of prosthetic heart valves in a heart of a patient. In some embodiments, methods and devices are presented for the treatment of valve disease by minimally invasive implantation of artificial replacement heart valves. In one embodiment, the artificial replacement valve can be a prosthetic valve device suitable for implantation and replacement of a mitral valve between the left atrium and left ventricle in the heart of a patient. In another embodiment, the prosthetic valve device can be suitable for implantation and replacement of an aortic valve between the left ventricle and the aorta in the heart of the patient. In further embodiments, the device can be suitable for implantation and repair or replacement of other heart valves, such as the tricuspid and pulmonary valves. FIG. 1 is a schematic illustration of a cross-sectional view of a heart depicting the major chambers, blood vessels, blood flow patterns and pertinent anatomical landmarks of the heart. FIG. 1 also shows an embodiment of an expandable prosthetic valve device 100 implanted in the native mitral valve region of the heart.

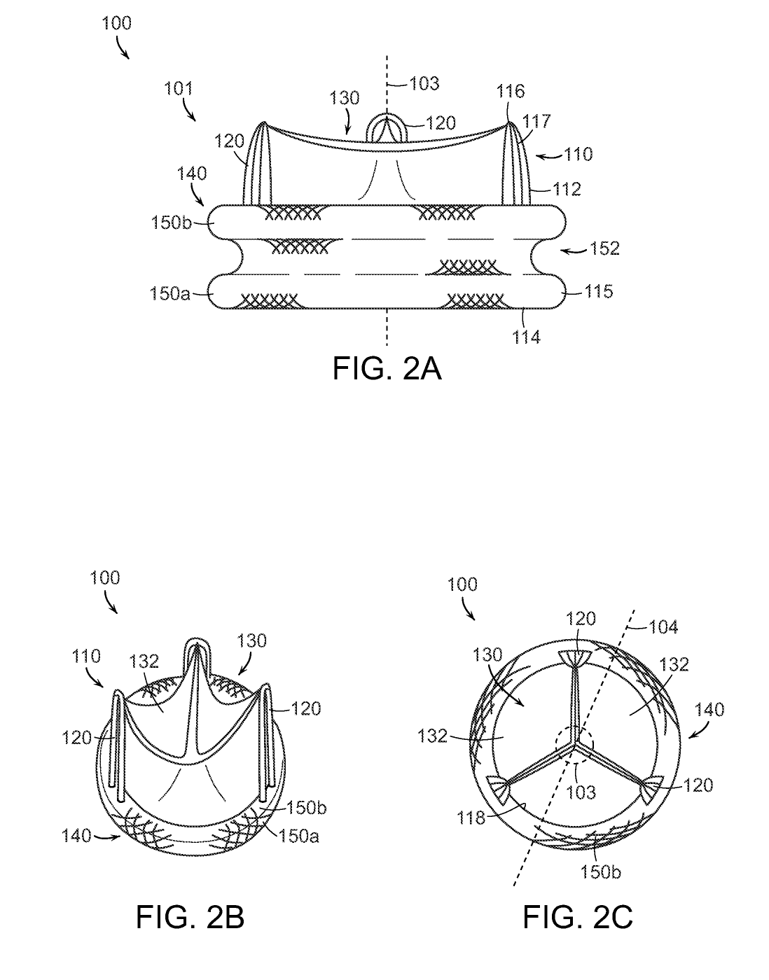

FIG. 2A is a side view of an expandable prosthetic valve device 100 for implantation at a native valve region of a heart, shown in a deployed state 101 (e.g., expanded configuration), and configured in accordance with an embodiment of the present technology. FIGS. 2B-2C are perspective and top views, respectively, of the device 100 as configured in FIG. 2A. Referring to FIGS. 2A-2C together, the prosthetic, valve device 100 can include an expandable structural support 110 and a self-expanding retainer 140 coupled to an outer surface 112 of the support 110. The structural support 110 can be generally cylindrical having a proximal end 114 at an upstream portion 115 and a distal end 116 at a downstream portion 117, the upstream and downstream portions 115, 117 oriented along a longitudinal axis 103 of the support 110 (FIG. 2A). Generally, when implanted, the upstream portion 115 of the device 100 is oriented to receive blood inflow from a first heart chamber (e.g., left atrium or left ventricle), and the downstream portion 117 is oriented to release blood outflow into a second heart chamber (or structure, e.g., left ventricle or aorta). A midline 104 (FIG. 2C), in a plane transverse to the longitudinal axis 103, divides the device 100 between the upstream and downstream portions 115, 117.

As shown in FIGS. 2A-2C, the support 110 can have an internal wall 118 and, in some embodiments, a valve structure 130 coupled to the internal wall 118 for governing blood flow through the prosthetic valve device 100. For example, the valve structure 130 can include a plurality of leaflets 132 (FIGS. 2B and 2C) that coapt and are configured to block blood flow through the prosthetic valve device 100 in an upstream direction (e.g., from the distal end 116 to the proximal end 114) and allow blood to flow through the device 100 in a downstream direction (e.g., from the proximal end 114 to the distal end 116). In some embodiments, the device 100 is configured for implantation at the native mitral valve and the device can have orifice or valve structure diameters varying between 23 and 33 mm. In other embodiments the device 100 may be suitable for valve replacement or repair of aortic, pulmonary and tricuspid valves having comparable valve structure diameters or smaller.

In one embodiment, the support 110 can be a flexible metal support 110 having posts 120, and the prosthetic valve structure 130 can be coupled to or otherwise supported by the posts 120. The plurality of leaflets 132 may be formed of various flexible and impermeable materials including PTFE, Dacron®, or biologic tissue such as pericardial tissue or xenograft valve tissue such as porcine heart tissue. In some embodiments, the valve structure 130 can include three leaflets 132; however, other embodiments may include two leaflet configurations or more than three leaflets 132.

In particular embodiments, the support 110 can be formed from a radially expandable cylindrical stent-like latticework of elastic material capable of being stored within a delivery catheter in a radially compressed state (e.g., delivery state, not shown) for delivery to a target valve site, and capable of being deployed to an expanded state 101 for deployment and implantation at the target valve site. In some embodiments, the support 110 can be a laser cut, fenestrated, nitinol or Elgiloy® tube. In one embodiment, the support 110 can be a balloon-expandable tubular metal stern with a tri-leaflet valve fashioned out of bovine pericardium, for example, mounted within the stent, such as the SAPIEN® Transcatheter Heart Valve (Edwards Lifesciences, Irvine, Calif.) or the CoreValve® (Medtronic, Minneapolis, Minn.). In embodiments that include a stented support 110, the thickness of the struts composing the framework of the stent could, in some examples, be less that about 0.75 mm, or in other examples, be between about 0.5 mm and 0.75 mm.

Stent-like supports 110 may be expanded, in some embodiments, by a radially expanding device such as a balloon or mechanical apparatus (not shown). In another embodiment, the support 110 can be self-expanding due to elasticity, superelasticity, shape memory or other responsive material behavior as described herein. The support 110 can include metals, polymers or a combination of metals, polymers or other materials. In some embodiments, the support 110 may be formed from either metallic tubes or sheet material. Some PCM processes for making similar structures are described in U.S. Pat. No. 5,907,893 by Zadno-Azizi, and in U.S. Patent Application 2007/0031584 by Roth, which are both herein incorporated in their entirety by reference. In some embodiments, components of the support 110 can include nickel-titanium alloys (e.g. nitinol), Elgiloy®, stainless steel, or alloys of cobalt-chrome. In other embodiments, components of the support 110 can include polymers such as Dacron®, polyester, polypropylene, nylon, Teflon®, PTFE, ePTFE, TFE, PET, TPE, PGA, PGLA, or PLA. Other suitable materials known in the art of elastic implants may be also be used to form some components of the support 110. In some arrangements, the support 110 can be formed at least in part from a cylindrical braid of elastic filaments as described further herein.

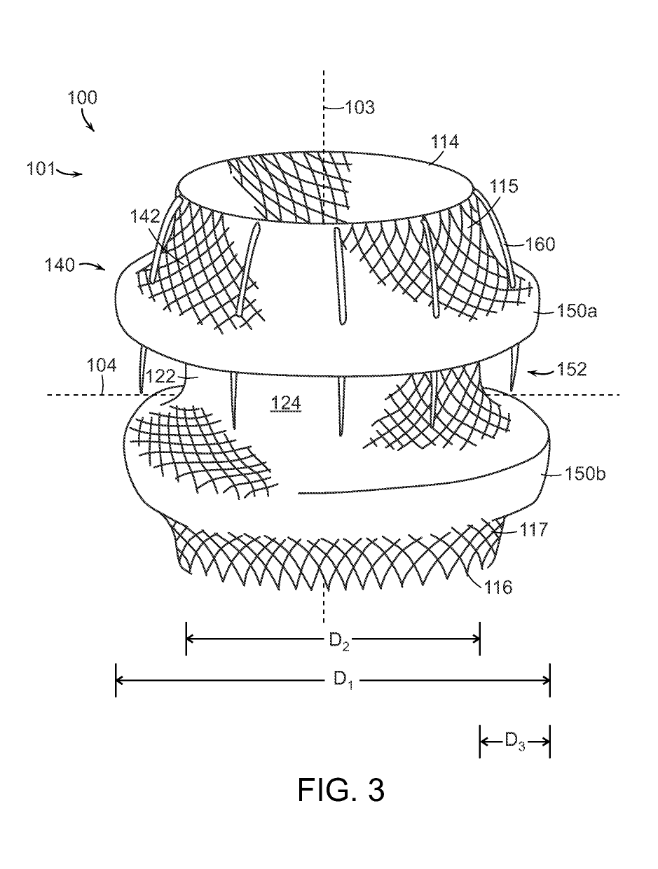

FIG. 3 is an isometric view illustrating an embodiment of another expandable prosthetic valve device 100 for implantation at a native valve region of a heart shown in a deployed state 101 (e.g., expanded configuration) and configured in accordance with an embodiment of the present technology. In the embodiment shown in FIG. 3, the retainer 140 can include an elastic, superelastic or other shape memory component that self-expands upon deployment of the device 100 to a formed or a pre-formed configuration at a target site. For example, the retainer 140 can include one or more braided or mesh layers that self-expand to a predetermined or pre-formed shape for providing a) a retaining feature for engaging a native heart valve annulus or other native tissue structure and/or b) a seal or occlusive property between the tissue and the outer surface 112 of the support 110 and/or the prosthetic valve structure 130 and the retainer 140. In one embodiment, the retainer 140 can expand from a delivery or compressed state (not shown) to a deployed state 101 (e.g., expanded configuration) having one or more annular flanges 150 (individually identified in FIGS. 2A, 213 and 3 as 150a and 150b). The annular flanges 150 can extend circumferentially around the support 110 for engaging a subarmular surface, supra-annular surface, or both sub-and supra-annular surfaces of a native valve annulus (e.g., mitral valve annulus or aortic valve annulus). In some embodiments, the device 100 can include a gap 152 (e.g., an annular recess) formed between the self-expanded flanges 150a and 150b that receives a native annulus or other anatomical tissue (e.g., native leaflets) of a heart.

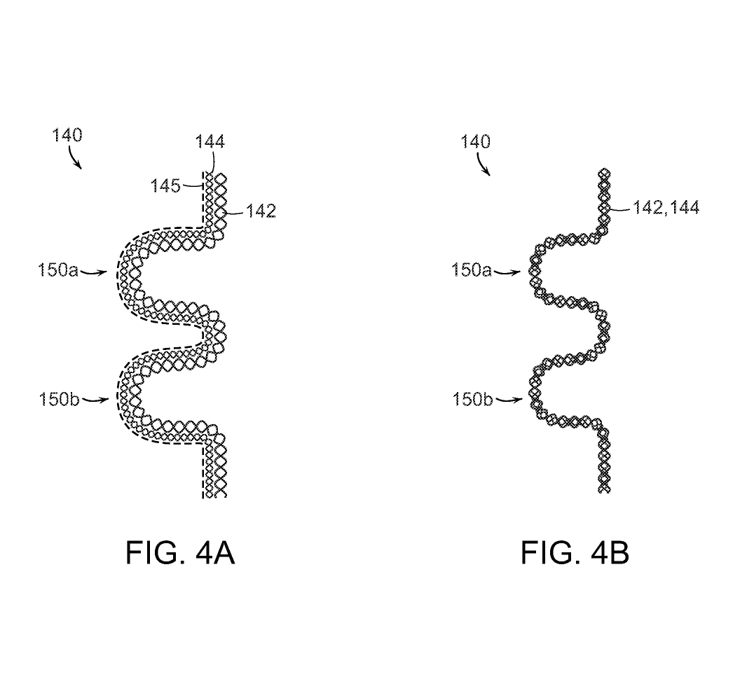

FIGS. 4A-4B are cross-sectional side views of portions of the retainer 140 configured in accordance with various embodiments of the present technology. In some embodiments the retainer 140 may consist of one or more braids, such as one or more structural braids 142 that define the shape and provide the primary expansion forces of the retainer 140. In another embodiment, the retainer 140 may include a structural braid 142 and a separate occlusive braid 144, as shown in FIG. 4A. In a different arrangement, the structural and occlusive braids 142, 144 can be combined into a single interwoven braid or braid layer that includes all the functions of both the structural and occlusive braids 142, 144 (FIG. 4B). Optionally, the retainer 140 may include other braids or layers in addition to the structure and occlusive braids 142, 144. For example, as shown in dotted line in FIG. 4A, a fabric or polymer layer 145 (e.g., comprising Dacron®, polyester, polypropylene, nylon, Teflon®, or other polymer, fabrics, braids, or knits) can be incorporated into the retainer 140 of the device 100.

The structural braid 142 can include one or more of a resilient material, shape memory material, or superelastic material such as Nitinol, for example. In the embodiments shown in FIGS. 2A-4B, the structural braid 142 expands to form the annular flanges 150a and 150b, separated by the gap 152. The annular flanges 150a, 150b can be regions of the retainer 140 that expand away from the support 110 to form the flange or “donut” feature around a circumference of the device 100.

The device 100 may be designed to fit within native valve regions of the heart, such as the native mitral or aortic valve regions. As shown in FIG. 3, the device 100 can have an overall expanded (or deployed) diameter D₁ at the annular flange 150a and/or 150b and the diameter D₂ at an intermediate portion 122, wherein the diameter D₁ is greater than the diameter D₂. The distance D₃ is defined by the distance that the annular flange 150 extends beyond the surface 124 of the intermediate portion 122. In some embodiments, the diameter D₂ can be approximately the same as the diameter of the target native valve at the annulus. In one embodiment, the second cross-sectional dimension D₂ can be approximately the same as a diameter of a heart valve region located at the annulus to accommodate the annulus of the native valve region within the gap 152. In other embodiments, the dimension D₂ could be greater or less than the diameter of the annulus as described further herein. In one embodiment, the dimension D₃ of the flange 1.50a and flange 150b can be sufficient to position the flanges 150a and 150b on the upper (e.g., upstream side) and lower (e.g., downstream side) surfaces of the native annulus, respectively to secure the device 100 to the native valve region (e.g., the annulus can fill the gap 152). In some embodiments, the diameter D₂ can range from about 20 mm to 40 mm. In some embodiments, the diameters may range from 23 mm to 35 mm. Although dimensions D₁-D₃ are described as diameters, heart valves, and in particular mitral valves, are not circular. Thus the retainer 140 can be thought of as having cross-sectional dimensions D₁-D₃ configured such that dimension D₁ is greater than that of the native valve annulus while dimensions D₂ is approximately equal to or slightly larger than the corresponding dimension of the native valve annulus. In some embodiments, the length of the device 100 from the proximal end 114 to the distal end 116 may range from 5 mm to 50 mm. In some embodiments, the length of the device may range from 10 mm to 40 mm.

In some embodiments, the device 100 may flex along its central longitudinal axis 103 to better conform to a native valve region or annulus of a native valve. In other embodiments, the device 100 may include annular flanges 150 or other protruding aspects from the support 110 through the self-expanding retainer 140 that have an irregular or non-cylindrical shape around. the support 110. In a specific embodiment, the device 100 may have an oval shape or deform to an oval shape or other shapes in the deployed state 101 to conform to the geometry of a native heart annulus and/or valve region. For example, the mitral valve, unlike the circular shape of the aortic valve, has an oval or kidney-like shape that may not be able to support conventional stents having a cylindrical configuration. Accordingly, the retainer 140 can expand to an irregular, non-cylindrical or, in some examples, oval-shaped configuration for accommodating mitral or other irregular shaped valves. Additionally, native valves (e.g., aortic, mitral) can be uniquely sized in patients and the device 100 for replacing such valves can be suitable for adapting to such size variations. For example, the overall circumference of the retainer 140 can expand and compress to conform to the unique size variations of the native annulus while maintaining its preformed curvilinear shape. In some instances, the present technology can be used to transform a conventional expandable stent, as described above, to the prosthetic valve device 100 described herein. For example, the retainer 140 can be coupled to a conventional stent (using the suturing or mechanical coupling techniques described herein or known in the art) to form the prosthetic valve device 100 with the sizing and shape adaptability functions described above.

In some arrangements, the occlusive braid 144 can be configured to provide for total or partial occlusion of blood around an outer region of the device 100 such that blood leakage between the valve structure 130 and/or the support 110 and the native tissue wall is inhibited from retrograde or backflow of blood from a downstream heart chamber to an upstream heart chamber. Accordingly, the occlusive braid 144 can function as a barrier to blood flow in those regions of the device 100 containing the occlusive braid 144. For devices 100 having a retainer 140 with an outer most braid or layer including or incorporating an occlusive braid 144, the occlusive braid 144 can provide a seal between the support 110, the structural braid 142 and/or any other component of the device 100 and the native tissue. In situations where the native tissue is uneven or varied across a tissue surface at a point of contact, the occlusive braid 144 can provide a seal that inhibits leakage of blood around or through the expandable support 110 in a downstream to upstream direction. Additionally, the occlusive braid 144 can, in some embodiments, provide a biocompatible scaffold to promote new tissue ingrowth and healing at the site of implantation.

In one embodiment, the support 110 can be formed from one or more structural 142 and/or occlusive 144 braids. In another embodiment, the support 110 can also be a radially expandable cylindrical stent-like latticework of elastic or superelastic material as described, above. In some embodiments, the support 110 and/or the retainer 140 may be formed using conventional machining, laser cutting, electrical discharge machining (EDM) or photochemical machining (PCM). Exemplary materials for the structural braid 142 and/or the occlusive braid. 144 include, but are not limited to nickel-titanium alloys (e.g. Nitinol), Elgiloy®, stainless steel, or alloys of cobalt-chrome. Materials may also include polymers such as Dacron®, polyester, polypropylene, nylon, Teflon®, PTFE, ePTFE, TIT, PET, TPE, PGA, PGLA, or PLA. Other suitable materials known in the art of elastic implants may also be used. In various embodiments, the materials used to form the structural braid 142 and the occlusive braid 144 can be the same or different. In further embodiments, the materials used to form the support 110 can be the same or different from the materials used to form either of the structure 142 or occlusive 144 braids.

In some embodiments, the structural braid 142 and/or the occlusive braid 144 can be formed at least in part from a cylindrical braid of elastic filaments. FIG. 5 is a side view of a mandrel 170 and a braided mesh or self-expanding braid 146 formed over the mandrel 170 configured in accordance with an embodiment of the present technology. The braid 146 may be formed over the mandrel 170 using techniques known in the art of tubular braid manufacturing. The resultant tubular braid 146 formed from any one of these processes may then be further shaped using a heat setting process. The braid 146 may be radially constrained without plastic deformation and can self-expand on release of the radial constraint. In some embodiments, the thickness of the braid filaments 148 used for forming the structural braid 142 would be less that about 0.5 mm. In some embodiments, the structural braid 142 may be fabricated from wires or filaments 148 having diameters ranging from about 0.015 mm to about 0.25 mm. In some embodiments, the thickness of the braid filaments 148 of the occlusive braid 144 are less that about 0.25 mm. In some embodiments, the occlusive braid 144 may be fabricated from filaments 148 having diameters ranging from about 0.01 mm to about 0.20 mm. The thickness (e.g., diameter) of the braid filaments 148 can be less than about 0.2 mm. In farther embodiments, the structural 142 and/or occlusive braids 144 can comprise braids having mixed filament diameters (e.g., thickness). For example, any braid of the retainer 140 (or the support 110) may be fabricated from filaments having a plurality of diameters ranging from about 0.015 mm to about 0.15 mm.

In some embodiments, at least the occlusive braid 144 may comprise metal filaments 148 that are less thrombogenic than commonly used polymeric medical fabrics such as polyester or Dacron®. In other embodiments at least the outer surface of the support 110 and/or annular flange 150 have filaments 148 that are less thrombogenic. In some embodiments, the metal filaments 148 may be highly polished or surface treated to further improve their hemocompatibility. In some embodiments, low thrombogenicity may provide a clinical advantage of lower thromboembolic bolic risk for the patient after device implantation.

In various arrangements, characteristics of the occlusive braid 144 and/or the angular flange 150 such as blood occlusion and promotion of tissue ingrowth can be in influenced by the “pore size” or “weave density” of the material. FIG. 6 is an enlarged view of a self-expanding braid with interwoven large 149 and small 147 strands configured in accordance with an embodiment of the present technology. As illustrated in FIG. 6, an effective pore size 180 is the largest “circle” that will fit within any individual cell of the braid 148. Pore sizes in the range of about 0.025 mm to 2.0 mm may be utilized in some embodiments. In other embodiments, the pore size 180 may be in the range of about 0.025 mm to about 0.30 mm. In another embodiment, pore sizes can be from about 0.10 mm to 2.0 mm, and in a further embodiment, pore sizes may be in the range of about 0.20 mm to about 0.75 mm or from about 0.05 mm to about 0.50 mm, or from about 0.10 mm to about 0.30 mm. In one embodiment the structural braid 142 can have a first pore size and the occlusive braid 144 can have a second pore size less than the first pore size. For example, the first pore size, in some embodiments can be about 0.50 mm to about 2.0 mm and the second pore size can be about 0.025 to about 0.30. In some embodiments, the retainer 140, the occlusive braid 144 or regions of braid forming the annular flange 150 can have braided filaments having pore densities ranging between 25-75%.

Referring back to FIG. 2A, the self-expanding retainer 140 can be coupled to, or in other arrangements, can be integral with the support 110. In one embodiment, the retainer 110 is attached to the support 110 using various suture or other attachment mechanisms known in the art. Examples of suture materials can include polyester, polypropylene, nylon or other suitable polymeric materials such as Dacron®, polyester, polypropylene, nylon, Teflon®,PTFE, ePTFE, TFE, PET, TPE, PGA, PGIA, or PLA. The retainer 140 can, in some embodiments, be a braided cylindrical tube configured to radially encompass or surround at least a portion of the support 110. In other embodiments, the retainer 140 can include other shaped structures or strips configured to be attached to portions the outer surface 112 of the support 110. In further embodiments, the device 100 can include a plurality of retainers 140 coupled to the support 110.

In some embodiments, the retainer 140 can include one or more layers of braid 148 disposed along the length of the device 100 from the proximal end 115 to the distal end 117 (FIG. 3), or in other embodiments, along a partial length of the device 100 (FIG. 2A) or intermittently along the length of the device 100. In some embodiments, braid filaments 148 of varying diameters may be used in different braids, such as illustrated in FIG. 6. In some embodiments, braid filaments 148 of varying diameters (small 147 and large 149 strands) can be combined in the same braid (such as shown in FIG. 6) or portions of the braid to impart different characteristics including: stiffness, elasticity, structure, radial force, pore size, occlusion ability, etc. In some embodiments, regardless of filament diameter, the braided filament 148 count for the occlusive braid 144 is greater than 290 filaments per inch. In one embodiment, the braided filament 148 count for the occlusive braid 144 is between about 360 to about 780 filaments per inch, or in further embodiments between about 150 to about 290 filaments per inch. In one embodiment, the braided filament 148 count for the structural braid 142 is between about 72 and about 144 filaments per inch, or in other embodiments between about 72 and about 162 filaments per inch. In other embodiments, the braided filament 148 count for any braid layer in the retainer 140 can be between 48 and 1600 filaments per inch. Or in other embodiments between 96 and 1200 filaments per inch. In further embodiments, the braided filament count is between 144 and 800 filaments per inch. In some embodiments, the device 100 may include polymer filaments 148 or fabric within the braid 146 or between layers of braids 142, 144.

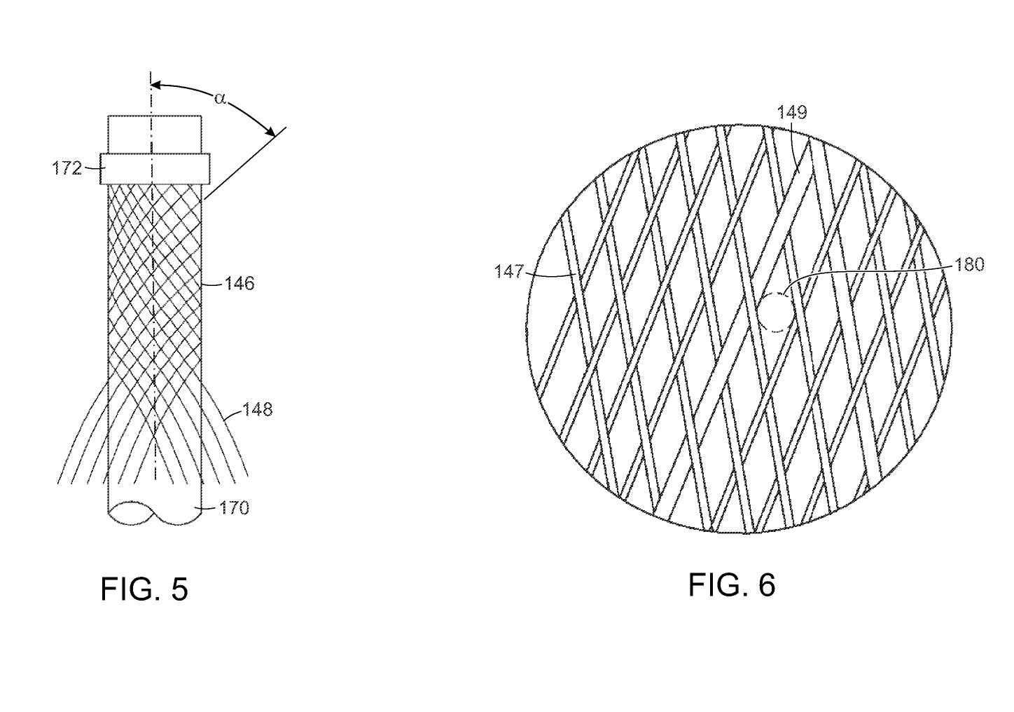

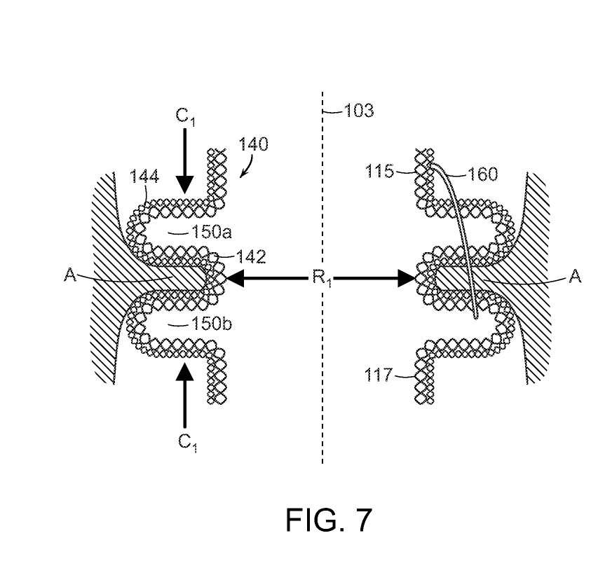

FIG. 7 is an enlarged cross-sectional side view of select components of an expandable prosthetic valve device 100 implanted at a native valve annulus A in the heart in accordance with an embodiment of the present technology. As shown in FIG. 7, the retainer 140 can include the structural braid 142 having sufficient resiliency and strength to provide a radial force R₁ in an outward, radial direction (in the direction of arrows R₁) from the longitudinal axis 103. Further, the structural braid 142 can engage the annulus A from an upstream surface (supra-annular surface) and from a downstream surface (sub-annular surface) and apply a compressive force C₁ against the upstream and downstream surfaces with the annular flanges 150a, 150b, respectively. The radial force R₁, the compressive force C₁, or a combination of radial R₁ and compressive C₁ forces can function to maintain the position of the prosthetic valve device 100 at the annulus of the native valve (e.g., mitral valve, aortic valve, tricuspid valve, pulmonary valve) to be repaired or replaced even under high blood pressure during systole (e.g., normal contraction of the left ventricle).

In one embodiment, the retainer 140 can apply compressive forces C₁ on the annulus A or other valve tissues (e.g., leaflets) while not applying a radial force R₁ (e.g., the radial force R₁ can be about zoo). In another embodiment, the radial force R₁ can be minimal, while the compressive force C₁ can function to maintain the desired position of the device 100 at the native valve region. For example, the retainer 140 can provide a compressive force C₁ that is greater than the radial force R₁. In some embodiments, the support 110 can have a cross-sectional dimension less than a corresponding dimension of the native valve region (e.g., the annulus A) such that any radial force R₁ applied by the device 100 against the native valve tissue is provided solely by the retainer 140. Thus, the radial force R₁ provided by the retainer 140 and/or the structural braid 142 can be less, or in some instances greater, than a corresponding radial force of the support 110 in the expanded configuration.

In some embodiments, the braids 142 and 144 of the flange 150 may be fabricated generally flat at the surfaces contacting the supra-armular and subannular surfaces of the tissue annulus, or optionally, the flange 150 may be fabricated in a serrated, scalloped or “wavy washer” fashion at the surfaces contacting the tissue annulus to increase compression and torsional stability. The terms “formed”, “preformed” and “fabricated” may include the use of molds or tools that are designed to impart a shape, geometry, bend, curve, slit, serration, scallop, void, hole in the elastic, superelastic, or shape memory material or materials used in the components of the valve 130, including the valve support 110 or annular flange 150. These molds or tools may impart such features at prescribed temperatures or heat treatments.

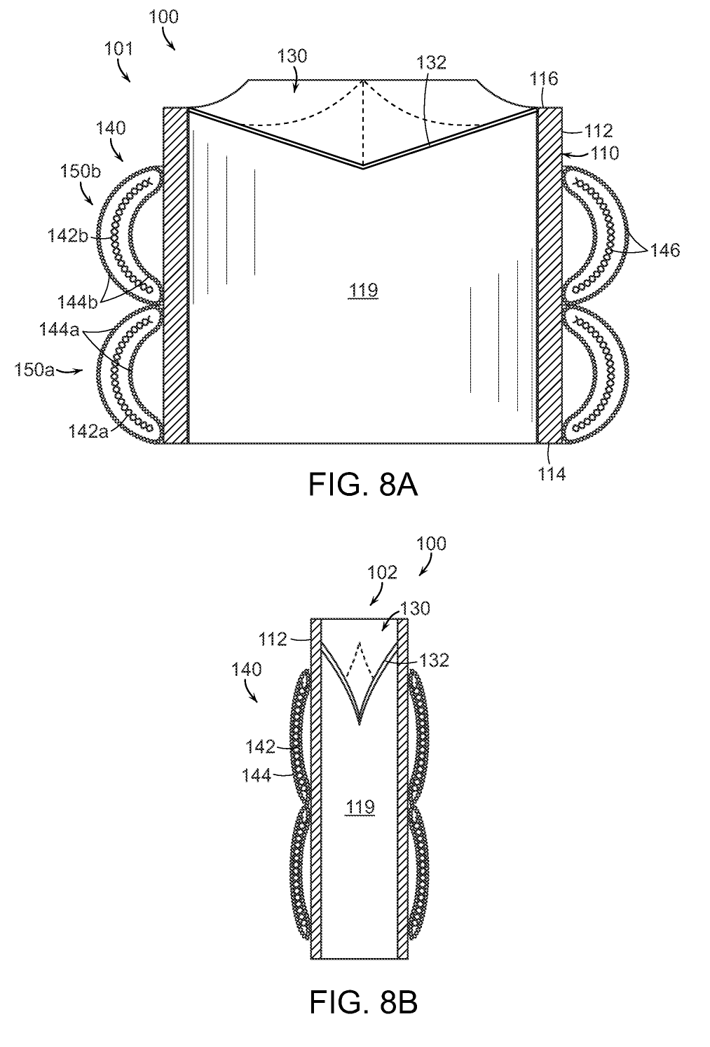

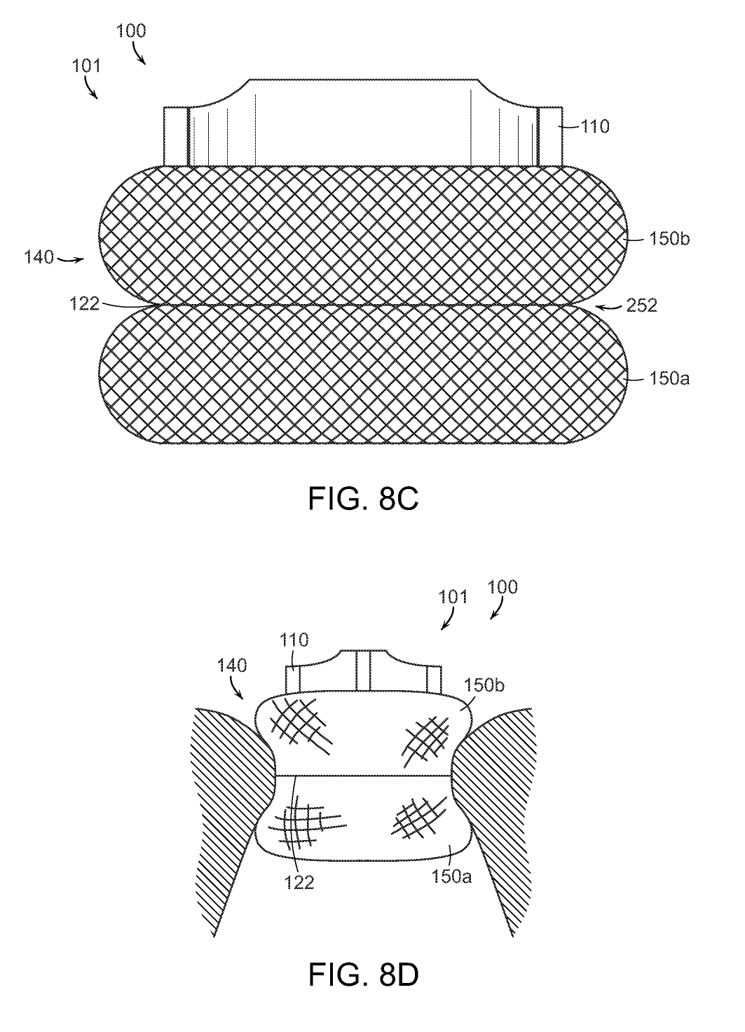

FIGS. 8A and 8B are enlarged cross-sectional views of an expandable prosthetic valve device 100 shown in a deployed state 101 (e.g., expanded configuration, FIG. 8A) and in a delivery state 102 (e.g., contracted configuration, FIG. 8B) configured in accordance with an embodiment of the present technology. FIG. 8A shows the device 100 having the expandable structural support 110 shown in an expanded configuration and an artificial valve 130 coupled to an interior portion 119 of the support 110. In one embodiment, the retainer 140 is coupled to the outer surface 112 of the support 110 and comprises a plurality of layers of self-expanding braided material 146 or mesh. As shown in FIG. 8A, the retainer 140 can include a plurality of different retainer portions (shown individually as 140a and 140b) coupled to the outer surface 112. Each retainer portion 140a, 140b is positioned for self-expansion into annular flanges 150a, 150b. While two retainer portions 140a, 140b are shown in FIG. 8A, one of ordinary skill in the art will recognize that more than two or, alternatively, a single retainer portion 140 could be coupled to the outer surface 112 of the support 110 and include superelastic and/or shape memory materials preformed or molded to form a plurality of flanges, such as flanges 150a and 150b, upon release from radial constraint and as shown in FIGS. 2A-3.

As discussed above, each retainer portion 140a, 140b) can include one or more structural braids 142 (shown independently in FIG. 8A as 142a and 142b) and can include one or more occlusive braids 144 (shown independently in FIG. 8A as 144a and 144b) covering and/or surrounding each of the structural braids 142a, 142b (referred to collectively as 142). Structural braids 142, as discussed above, can be an elastic, superelastic or other shape memory material that self-expands upon deployment of the device 100 to a formed or a pre-formed configuration. The structural braid 142 can include one or more of a resilient material, shape memory material, or superelastic material such as nickel-titanium alloys (e.g. Nitinol), Elgiloy®, stainless steel, or alloys of cobalt-chrome. Materials may also include polymers such as Dacron®, polyester, polypropylene, nylon, Teflon®, PTFE, ePTFE, TFE, PET, TPE, PGA, PGLA, or PLA. Additionally, and as discussed with reference to FIG. 7, the structural braid 142, while being resilient and conformable to surrounding native valve tissue, can provide radial R₁ and/or compressive C₁ forces against native valve tissue (e.g., the annulus) that prevents the prosthetic valve device 100 from becoming dislodged from the annulus of the native valve during normal ventricular contraction (e.g., systole).

In the illustrated embodiment, each retainer portion 140a, 140b can also include more than one occlusive braids 142a, 142b and/or an occlusive braided tube through which or under which one or more internal structural braids 142 can be received. In some embodiments the occlusive braid 144 can include braided filaments made from a variety of expandable and/or superelastic materials, such as nickel-titanium alloys (e.g. Nitinol), Elgiloy, stainless steel, or alloys of cobalt-chrome. Materials may also include polymers such as Dacron, polyester, polypropylene, nylon, Teflon, PTFE, ePTFE, TFE, PET, TPE, PGA, PGLA, or PLA. Other suitable materials known in the art of elastic implants may be used.

One of ordinary skill will recognize that other layering arrangements are possible, for example the structural braid 142 can be a braided tube that is fitted over and coupled to the support 110, and the occlusive braid 144 can be braided tube configured to fit over the structural braid 142. Additionally, structural braids 142 and occlusive braids 144 can be interspersed or arranged in layers in variety of manners over or around an outer surface 112 of the support 110.

The self-expanding retainer 140 can also be retained in a collapsed delivery state 102 (shown in FIG. 8B) such as when the retainer 140 and/or the device 100 is radially constrained, for example, within a delivery catheter sheath (not shown). In the delivery state 102, the retainer 140 can be elongated, folded or otherwise brought into close proximity to the outer surface 112 of the support 110. Upon release of the radial constraint, the self-expanding retainer 140 can self-expand to the deployed state 101 (FIG. 8A). Additionally, in the event that the prosthetic valve device 100 need to be repositioned, removed and/or replaced after implantation, the retainer 140 and the support 110 can transition from the deployed, state 101 (FIG. 8A) back to the delivery state 102 (FIG. 8B) using a catheter device or other lateral retaining sheath.

FIG. 8C is a side view of the expandable prosthetic valve device 100 as configured in FIG, 8A and FIG. 8D is a side view the expandable prosthetic valve device 100 of FIG. 8C implanted at a native aortic valve in the heart in accordance with an embodiment of the present technology. As shown in FIG. 8C, the device 100 includes a support 110 and a retainer 140 coupled to the support 110. In this embodiment, the retainer 140 expands to the deployed state having adjacent annular flanges 150a and 150b, wherein the adjacent annular flanges are separated by a gap 252 at the intermediate portion 122, and wherein the gap 252 is smaller than gap 152 as shown in the embodiments of FIGS. 2A-3.

FIG. 8D shows the device 100 implanted at the native aortic valve and positioned so that blood leaving the left ventricle can flow through the device 100 in an upstream to downstream manner. The retainer 140, having flanges 150a and 150b, provides placement and fixation of the device 100 at the aortic annulus. In some patients, the aortic annulus may be minimal in size and/or have congenital abnormalities and/or abnormal features resulting from disease. For example, the aortic annulus may, in some instances, be hardened due to calcification and aortic valve stenosis. The annulus and surrounding aortic valve tissues, including leaflets, may together be engaged by the retainer 140. Accordingly, the retainer 140 having braids (e.g., structural braid 112 and occlusive braid 144, shown in FIG. 8A) can provide both stabilizing forces and sealing capabilities (e.g., to inhibit paravalvular leaking) to the abnormal shape and features present in a specific patient's aortic valve.

Referring to FIG. 8D, the fully expanded circumference of the prosthetic valve device 100 and/or the presence or size of the gap 252 (FIG. 8C) at the intermediate portion 122 (shown in FIG. 3) may be selected in some instances to exceed the corresponding circumference of the native valve tissue, including the annulus and/or leaflets. Certain embodiments of devices having a larger circumference than a corresponding circumference of a native valve annulus, may increase the radial force R₁ (FIG. 7) of the retainer 140 after placement and help promote fixation of the device 100 to the annulus, and in some cases widening of the native tissue, especially in instances of valve narrowing (e.g., aortic valve stenosis). For example, in instances where the native aortic valve is hardened and/or calcified (e.g., aortic valve stenosis), retainers 140 applying radial force R₁ (FIG. 7) to this region in combination with compression force C₁ (e.g., to the annulus or to the native leaflets, shown in FIG. 7) can assist in promoting proper placement, fixation and retention of the device 100 to the native aortic valve region. In some embodiments, the maximum expansion of the retainer 140 is controlled to expand to the corresponding dimension of the native valve tissue. Other embodiments include devices 100 having retainers 140 that only apply compressive force C₁ to the native tissue and/or provide minimal radial force R₁ (FIG. 7). Such devices 100 may be useful for implantation at the native mitral valve or other valves, including a noncalcified aortic valve.

In addition to annular flanges and other expansion threes associated with the expandable prosthetic valve device 100, certain embodiments configured in accordance with the present technology can include one or more fixation members. Referring back to FIG. 3, some embodiments of the device 100 can include one or more fixation members 160 configured to provide additional fixation of the annular flange 150a and/or the device 100 to the native valve annulus. Fixation members 160 can include, for example, tines, barbs, hooks, pins or other anchors known in the art that can provide pressure or penetrating retention on the native annulus tissue when the device 100 is positioned in the deployed state 101 at the target valve. FIG. 3 shows a plurality of fixation members 160 generally attached to the upstream portion 115 of the device 100 and extending outward and in a downstream direction. As shown in FIG. 3, the fixation members 160 can be tines having a length from about 1 to 8 mm, or in another embodiment, about 4 to 6 mm. In one embodiment, shown in FIG. 3, the fixation members 160 can pierce a first annular flange 150a and extend partially into the gap 152 to apply retention pressure against an annulus when implanted at a native valve region, or to pierce or penetrate annular tissue to provide further retention of the device 100 to the annulus A. As illustrated in FIG. 7, the fixation member 160 can be configured to pass through the first annular flange 150a, pierce the annulus A and pass at least partially through the second annular flange 150b. Other embodiments of devices 100, not shown, could include fixation members that only partially pass through the first annular flange 150a. Additional embodiments can include devices 100 having one ore more fixation members affixed to a distal portion 117 of the device 100 and extending outward and in an upstream direction for passing through the second annular flange 150b and/or piercing the annulus A at a subannular surface. Such subannular arrangements of fixation members can be useful for maintaining the positioning of mitral valve replacement devices 100 during systole (e.g., ventricular contraction). In additional embodiments, fixation members 160 can include additional expandable wires or filaments, struts, supports, clips, springs, glues, adhesives or vacuum.

The fixation members 160 shown in FIG. 3 are generally evenly spaced around a circumference of the device 100; however, the fixation members 160 could be unevenly spaced or irregularly spaced around the circumference. For example, the device 100 can include fixation members 160 spaced in one or more groups generally aligned with regions of the native annulus coupled to native leaflets. A prosthetic heart valve device 100 configured to replace a native mitral valve may have two groups of fixation members 160, for example, generally aligned on the device 100 so as to engage portions of the annulus attached to the anterior and posterior leaflets. Regions of the annulus retaining leaflets or remnants of leaflets may be thicker or otherwise have a varied profile with respect to the remainder of the annulus tissue, and fixation members 160 can be used to press into, penetrate or otherwise grasp tissue in these and/or other areas during implantation. Alternatively, fixation members 160 can be generally aligned on the device 100 so as to engage other portions of the annulus such as adjacent or near the native anterolateral commissure and posteromedial commissure. In additional embodiments, the device 100 may include a combination of different types of fixation members 160 disposed circumferentially around the upstream portion 122 or other portion of the device 100.

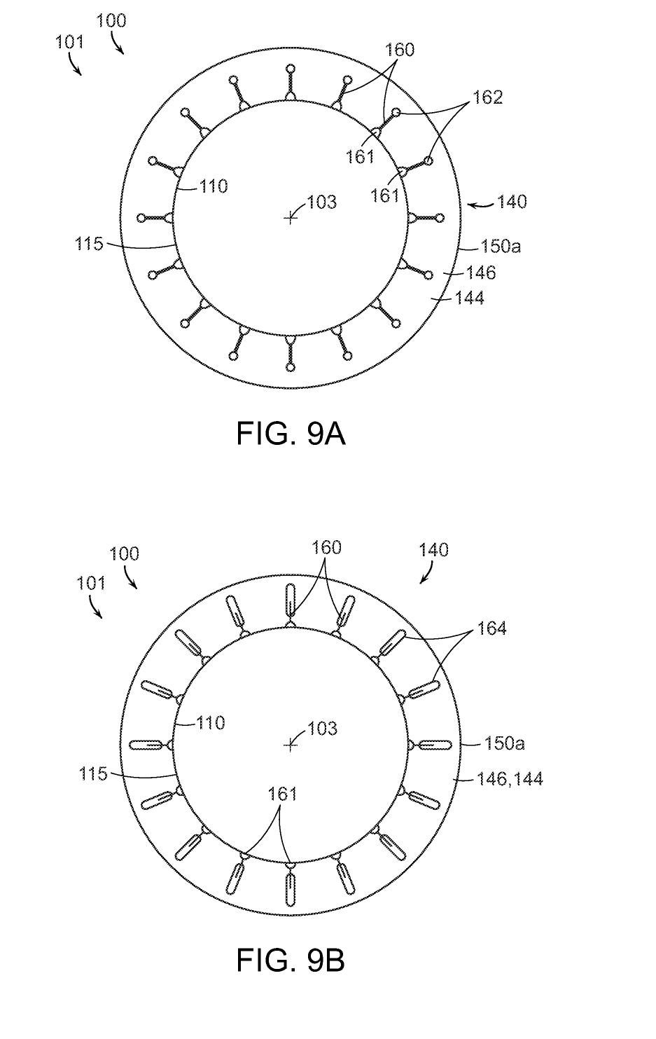

FIGS. 9A-9B are top views of expandable prosthetic valve devices 100 having a plurality of fixation members 160 configured in accordance with embodiments of the present technology. As shown in FIGS. 3 and 9A, the fixation members 160 can be coupled to the support 110, for example at an upstream portion 115, at attachment points 161 and extend outwardly in a downstream direction from the attachment points 161. In one embodiment, the fixation members 160 can pass through at least one layer of braid material 146, such as the occlusive braid 144, of the annular flange 150 (e.g., the first annular flange 150a) at braid puncture points 162. In additional arrangements, the fixation members 160 can pass through one or more braids of the retainer 140 comprising the annular flange 150 (e.g., the first annular flange 150a). In further embodiments, and as discussed above, the fixation members 160 can have a length and rigidity sufficient to pierce the annular tissue, penetrate through the annulus A, and/or pass through at least an initial braid (e.g., occlusive braid 144) of the second flange (not shown). In another embodiment, the fixation members 160 can press on the retainer 140 of the annular flange 150, such as the first annular flange 150a, to increase the compressive force C₁ for engaging the annulus A (FIG. 7). Referring to FIG. 9B, certain embodiments of prosthetic valve devices 100 can have annular flanges 150 with preformed. slots 164, slits or holes through one or more curvilinear portions of the flange 150. For example, the fixation members 160 can pass through the initial braid of the flange 150 (e.g., the first annular flange 150a) through the preformed slots 164 (e.g., slits or holes) circumferentially disposed in portions of the braid 148 around a circumference of the flange 160 and that are aligned with the fixation members 160.

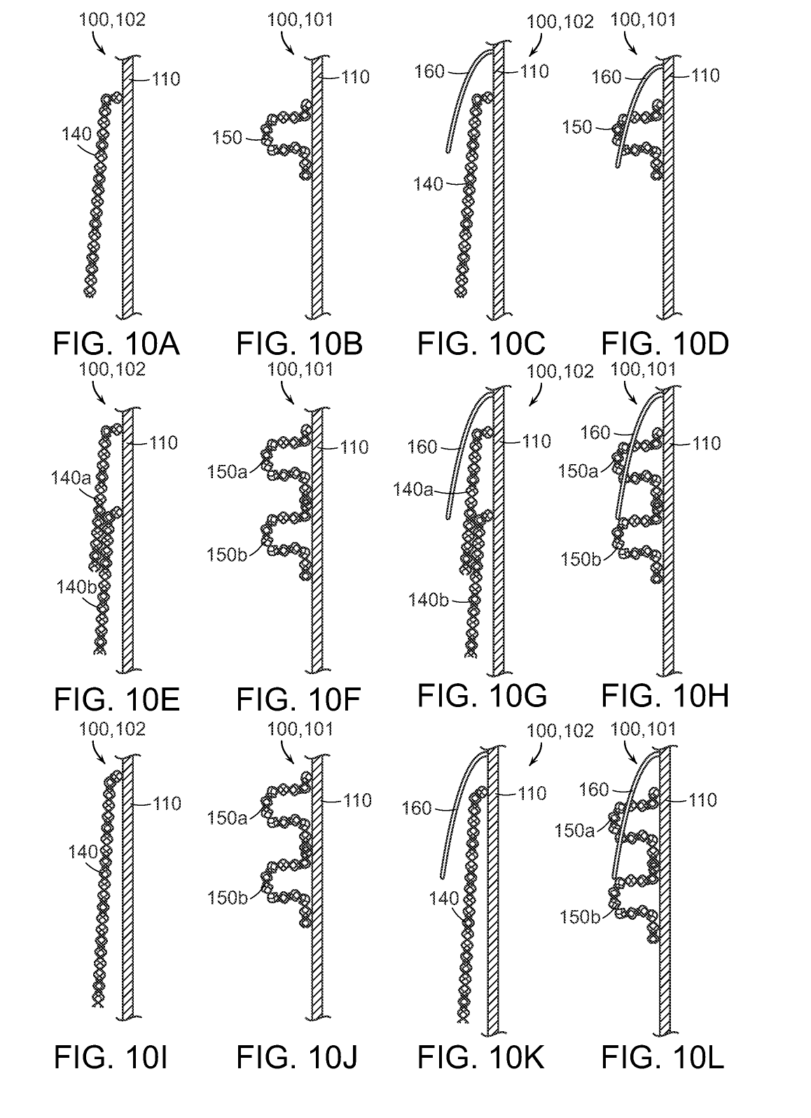

FIGS. 10A-10L are cross-sectional side views of portions of expandable prosthetic valve devices 100 in delivery 102 and deployed 101 states configured in accordance with various embodiments of the present technology. The cross-sectional views shown in FIGS. 10A-10L show variations in retainer 140 arrangements and illustrate how the variable retainers 140 transition from the delivery state 102 (e.g., linear or contracted configuration) to the deployed state 101 (e.g., curvilinear or expanded state). The various devices depicted can have a support 110 and one or more retainers 140 coupled to the support 110. The retainers 140 can include interwoven braid layers, such as interwoven structure 142 and occlusive braids 144, as shown in the embodiments illustrated in FIGS. 10A-10L; however, it is understood that the retainer 140 can include separate braids 142, 144 as well as a combination of multiple braids 142, 144 and other materials (e.g., optional fabric or polymer layer 145, FIG. 4A).

FIGS. 10A and 10E, for example, show a support 110 having a retainer 140 in a linear/contracted configuration (such as within a catheter sheath, not shown) coupled to the support 110, and FIGS. 10B and 10J show the retainers 140 in the expanded state (e.g., catheter/radial contraction, not shown, removed) having a single flange 150 (FIG. 10B) and having two flanges 150a and 150b (FIG. 10J). Alternatively, and as shown in FIG. 10B, two or more retainer portions 140a and 140b can be coupled to the support 110. FIG. 101F shows the two retainer portions 140a and 140b in expanded configurations as annular flanges 150a and 150b.

The support 110 can also include one ore more fixation member 160 as discussed above. FIGS. 10C, 10G and 10K show similar cross-sectional views to FIGS. 10A, 10E and 10I, respectively, however, in the embodiments shown in these figures, the support 110 includes the fixation member 160. FIGS. 10D, 10H and 10L show expanded configurations for the devices 100 illustrated in FIGS. 10C, 10G and 10K, respectively, and show the fixation member pass through or penetrate portions of the expanded annular flange 150 (e.g., flange 150a in FIGS. 10H and 10L).

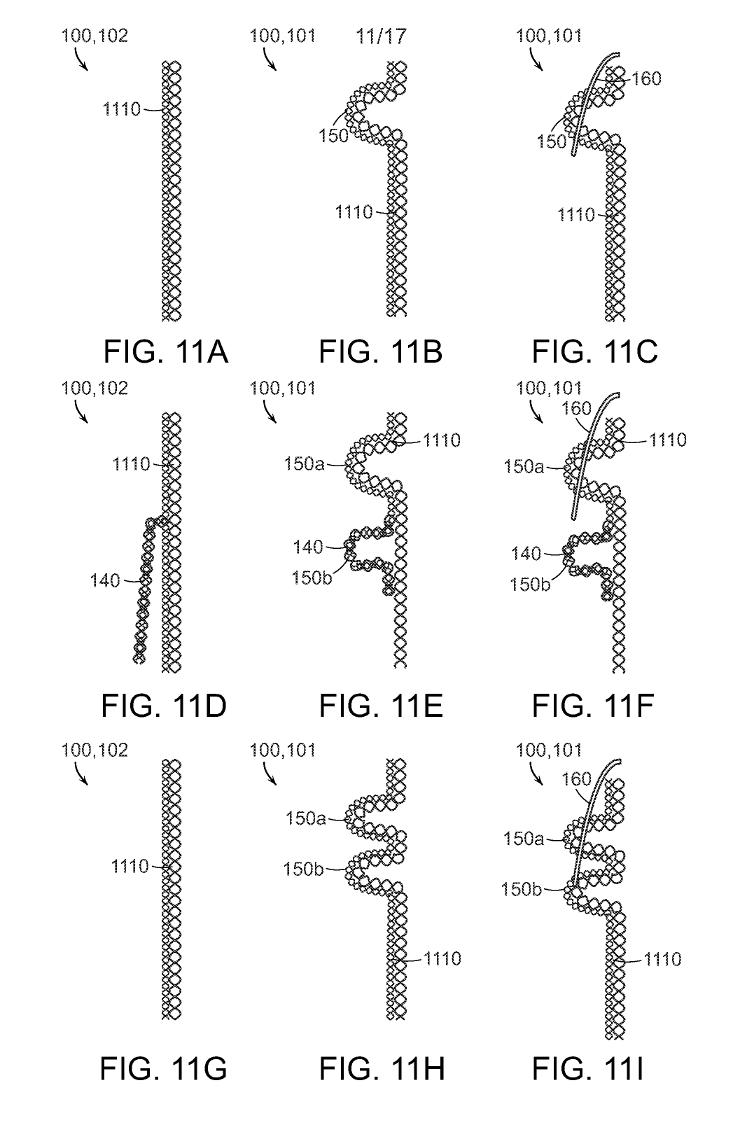

FIGS. 11A-11I are cross-sectional side views of portions of expandable prosthetic valve devices in delivery 102 and deployed 101 states configured in accordance with additional embodiments of the present technology. The cross-sectional views shown in FIGS. 11A-11I show variations in a support 1110 and retainer 140 arrangements and illustrate how the variable support 1110 and retainers 140 transition from the delivery 102 (e.g., linear or contracted configuration) to the deployed 101 states (e.g., curvilinear or expanded state).

As shown, the support 1110 of FIGS. 11A-11I can include a self expanding braid and/or, in some embodiments, be a component of the retainer 140 such that the structural rigidity and resilience appropriate for deploying and anchoring a replacement heart valve can be provided by the braided support 1110, the retainer 140 or a combination of the braided support 1110 and retainer 140. In some embodiments, the valve structure 130 (FIGS. 2A-2C) can be directly coupled to the self-expanding retainer 140 or the braided support 1110 without the posts 120 (FIGS. 2A-2C) or stent-like latticework of the commercially available percutaneous heart valves described above. In any case, the valve structure 130 will be collapsible so as to have a profile suitable for percutaneous delivery, and be expandable with the braided support 1110 and/or retainer 140 for implantation at the native valve location.

In some arrangements, the device 100 will not have a separate support 110 but will have a braided support 1110 (FIGS. 11A, 11D and 11G), and the functionality of the support 110 described with respect to other embodiments (e.g., FIGS. 2A-2C) is provided by the braided support 1110 and, in some embodiments, by at least a portion of the retainer 140. The braided support 1110 can transition to a deployed state 101 to provide a single (FIG. 11B and 11E) annular flange 150 or to provide multiple (FIG. 11H) annular flanges 150a and 150b. FIGS. 11D and 11E illustrate an example where additional retainers 140 can be coupled to the braided support 1110 to provide additional curvilinear features to the device 100 in the deployed state 101, FIGS. 11C, 11F and 11F show examples of devices 100 having a braided support 1110 and an additional fixation member 160 attached to the braided support 1110 and in the deployed state 101.

### Additional Embodiments of Prosthetic Valve Device Retainers and Braids

In some embodiments, the filaments of the braided mesh can be generally in an axially elongated configuration within a delivery catheter. In some embodiments, the filaments are more parallel with the filament braid angle “α” as shown in FIG. 5, e.g., between about 0 and 45 degrees with respect to the central longitudinal axis 103 of the device 100. In some embodiments, the filaments 148 of any one of the braids 142, 144 of the retainer 140 in the expanded/deployed configuration 101 (e.g., not within a delivery catheter) are more perpendicular, for example, with a between about 45 and 90 degrees with respect to the central longitudinal axis 103 of the device 100.

In some embodiments, the retainer 140 conforms to the native valve region without annular flanges 150 along the central longitudinal axis 103. In such embodiments, expanded diameters can range from about 20 mm to 60 mm. In other embodiments, expanded diameters can range from about 25 mm to 35 mm. In some embodiments, the diameters of the retainer 140 within the delivery catheter (e.g., in the delivery state 102, FIG. 8B) range from about 1 mm to 10 mm, and in other embodiments, range from about 1.5 mm to 5 mm.

In some embodiments, filler, sealing, bonding agents including hydrogel may be incorporated into the device 100 components such as the structural braid 142 or occlusive braid 144 of the retainer 140 to improve neck sealing and/or occlusion.

For some embodiments, certain braid characteristics can be valuable for a woven or braided prosthetic valve device 100 that can achieve a desired clinical outcome for repair or replacement of a native heart valve. For example, it may be desirable, in some instances, for the device 100 and/or the braided portion 140 to have sufficient radial stiffness for stability, limited pore size for rapid promotion of hemostasis leading to occlusion, and a collapsed profile which is small enough to allow insertion through an inner lumen of a vascular catheter. A retainer 140 with a radial stiffness below a certain threshold may be unstable and may be at higher risk of movement or embolization in some cases. Larger pores between filament intersections in a braided or woven structure may not generate thrombus and occlusion in an acute setting and thus may not give a treating physician or health professional clinical feedback that the flow disruption will lead to a complete and lasting occlusion of blood flow in areas around the valve structure 130 and/or between the valve structure 130 and the native valve tissue. Delivery of a device 100 for treatment of a patient's vasculature through a standard vascular catheter may be highly desirable to allow access through the vasculature in the manner that a treating physician is accustomed. The maximum pore size in a portion of a device 100 (e.g., a retainer 140) that spans the native annulus is desirable for some embodiments of a device 100 having a retainer 140 for treatment and may be expressed as a function of the total number of all filaments, filament diameter and the device diameter. The difference between filament sizes, where two or more filament diameters or transverse dimensions are used, may be ignored in some cases for devices 100 where the filament size(s) are very small compared to the device dimensions. For a two-filament device, the smallest filament diameter may be used for the calculation. Thus, the maximum pore size for such embodiments may be expressed as follows:

Pmax=(1.7/NT)(pD−(NTdw/2))
where Pmax is the average pore size,
D is the Device diameter (transverse dimension),
NT is the total number of all filaments, and
dw is the diameter of the filaments (smallest) in inches.

Using this expression, the maximum pore size, Pmax of the of one or more braids (e.g., braids 142, 144) of the retainer 140 may be less than about 0.016 inches or about 400 microns for some embodiments. In some embodiments the maximum pore size of one or more braids of the retainer 140 may be less than about 0.012 inches or about 300 microns.

The collapsed profile of a two-filament (profile having two different filament diameters) braided filament layer (e.g., structural braid 142 or occlusive braid 144) may be expressed as the function:

p_{c}=1.48 ((Nₗdₗ²+N^{s}dₛ²))^{1/2}
where Pc is the collapsed profile of the braid,
Nl is the number of large filaments,
Ns is the number of small filaments,
dl is the diameter of the large filaments in inches, and
ds is the diameter of the small filaments in inches.

Using this expression, the collapsed profile Pc may be less than about 1.0 mm for some embodiments of a braid such as the occlusive braid 144. In some embodiments, the device 100 may be constructed so as to have a braid with both factors (Pmax and Pc) described above within the ranges descrubed; Pmax less than about 300 microns and Pc less than about 1.0 mm. In some such embodiments, the braid may include about 70 filaments to about 300 filaments. In some cases, the filaments may have an outer transverse dimension or diameter of about 0.0005 inches to about 0.012 inches.

In some embodiments, a combination of small and large filament sizes may be utilized to make a device with a desired radial compliance and yet have a collapsed profile which is configured to fit through an inner lumen of commonly used vascular catheters. A device fabricated with even a small number of relatively large filaments can provide reduced radial compliance (or increased stiffness) compared to a device made with all small filaments. Even a relatively small number of larger filaments may provide a substantial increase in bending stiffness due to change in the moment of inertia that results from an increase in diameter without increasing the total cross sectional area of the filaments. The moment of inertia (I) of a round wire or filament may be defined by the equation:

I=πd⁴

where d is the diameter of the wire or filament.

Since the moment of inertia is a function of filament diameter to the fourth power, a small change in the diameter greatly increases the moment of inertia. Thus, a small change in filament size can have substantial impact on the deflection at a given load and thus the compliance of the device 100.

Thus, the stiffness can be increased by a significant amount without a large increase in the cross-sectional area of a collapsed profile of the device 110 (shown in FIG. 6E). As such, some embodiments of devices for treatment of a patient's vasculature may be formed using a combination of filaments with a number of different diameters such as 2, 3, 4, 5 or more different diameters or transverse dimensions. In device embodiments where filaments with two different diameters are used, some larger filament embodiments may have a transverse dimension of about 0.004 inches to about 0.012 inches and some small filament embodiments may have a transverse dimension or diameter of about 0.0005 inches and about 0.003 inches. The ratio of the number of large filaments to the number of small filaments may be between about 4 and 16 and may also be between about 6 and 10. In some embodiments, the difference in diameter or transverse dimension between the larger and smaller filaments may be, in some embodiments, less than about 0.008 inches, in other embodiments, less than about 0.005 inches, and in further embodiments, less than about 0.003 inches.

For some embodiments, it may be desirable to use filaments having two or more different diameters or transverse dimensions to form a permeable shell in order to produce a desired configuration (e.g., an annular flange 250) as discussed in more detail below. The radial stiffness of a two-filament (two different diameters) braid (e.g., structural braid 142) may be expressed as a function of the number of filaments and their diameters, as follows:

S_{radial}=(1.2×10⁶ lbf/D⁴)(Nₗdₗ⁴+Nₛdₛ⁴)

where S_{radial} is the radial stiffness in pounds force (lbf),
D is the Device diameter (transverse dimension),
Nₗ is the number of large filaments,
Nₛ is the number of small filaments,
dₗ is the diameter of the large filaments in inches, and
dₛ is the diameter of the small filaments in inches.

Using this expression, the radial stiffness, S_{radial} may be between about 0.014 and 0.284 lbf force for some embodiments.

In some embodiments, the radial stiffness near the proximal and distal ends 114, 116 as well as the intermediate portion 122 may be substantially greater than the radial stiffness of the regions encompassing the annular flanges 150a, 150b. Thus, the annular flanges 150a, 150b may be much more compliant than the proximal and distal ends 114, 116 and/or intermediate portion 122 allowing these flange regions to conform to anatomical variation at and around the annulus. Greater compliance may provide improved surface area contact and resistance to movement. In some embodiments, the radial stiffness of the intermediate portion 122 and/or near proximal and distal ends 114, 116 may be between about 1.5× and 5× the radial stiffness of the regions encompassing the annular flanges 150a, 150b.

### Further Embodiments of Prosthetic Valve Devices

FIG. 12A is a side view of an expandable prosthetic valve device 200 showing a self-expanding braid 246 transitioning from a delivery state to a deployed state and configured in accordance with an additional embodiment of the present technology. FIG. 12B is an enlarged cross-sectional side view of an expandable prosthetic valve device 200 shown in the delivery state 201 and resulting from the transition step illustrated in FIG. 12A. Referring to FIGS. 12A-12B together, the prosthetic device 200 includes features generally similar to the features of the prosthetic device 100 described above with respect to FIGS. 2A-11I. For example, the device 200 can include the support 110 and have the retainer 140 coupled to the support 110. However, in the embodiment shown in FIGS. 12A-12C, the braids (e.g., structural 142 and occlusive 144 braids) evert and roll in an inside-out fashion to form circular, rolled (e.g., toroidal) annular flanges 250a, 250b (shown in FIG. 12B).

As shown in FIG. 12A, the retainer portions (shown independently in FIG. 12A as 140a and 140b) can include a plurality of stacked first and second retainer portions 140a, 140b coupled to the support 110 at attachment sites 141a and 141b on upstream and downstream portions 115, 117 of the support 110, respectively. During the transition from delivery to deployment states, the first retainer portion 140a can be released from restraint and roll back onto itself in an inside-out fashion to form the rolled toroidal-shaped annular flange 250a at the upstream portion 115 of the support 110 (shown in dotted lines in FIG. 12A). Once the first retainer portion 140a transitions to the expanded configuration, the second retainer portion 140b, retained under the first retainer portion 140a, is released and can roll back onto itself in an inside-out fashion to form the rolled toroidal-shaped annular flange 250b at the downstream portion 117 of the support 110 (shown in FIG. 12B). In one embodiment, the first and second retainer portions 140a, 140b can evert to form single layered toroidal annular flanges 250a, 250b as shown in FIG. 12B. Alternatively, the first and second retainer portions 140a, 140b can evert in a tighter or more compact rolled manner to form multilayered toroidal annular flanges 250a, 250b (not shown).

In another embodiment, FIG. 12C shows a device 200 having an elongated retainer 240 that surrounds the outer surface 112 of the support 110 and extends beyond a length of the support 110 such that, upon deployment, the elongated sections of the elongated retainer 240 evert from proximal and distal ends 441 and 443 and roll in an inside-out fashion (in a roll direction opposite from the roll direction shown in FIGS. 12A-12B) to form toroidal annular flanges 250a and 250b.

FIG. 12D is a cross-sectional side view of the prosthetic valve device 200 of FIG. 12C implanted at a native mitral valve in the heart in accordance with an embodiment of the present technology. As shown, the toroidal annular flanges 250a, 250b can position around and against the native annulus A. thereby providing both radial and compressive forces against the native annulus A. The toroidal annular flanges 250a, 250b have a cross-sectional dimension D₄ (shown in FIG. 12C) greater than a corresponding dimension D_{A} (shown in FIG. 12D) of the native valve region. As such, when the device 200 is positioned and expanded at the annulus A (e.g., during deployment), the annular flanges 250a, 250b will compress inwardly from the original circular shape and expand around the shape of the native annulus. In so doing, the toroidal annular flanges 250a, 250b can form at tight coupling at the native annulus A, and form a seal between the support 110 and the native tissue.

While the device 200 is shown implanted at a native mitral valve in the heart, it will be understood that any of the devices 100, 200 described herein can be configured and deployed at the native aortic valve or other heart valves (e.g., tricuspid, pulmonary). Indeed, the native aortic valve annulus and surrounding tissue can, in certain disease states, provide difficult, hard (or soft) and uneven surfaces to engage with conventional valve replacement devices and stents. The devices, 100, 200 described herein, in certain embodiments, can provide annular flanges 150, 250 and other retainers 140 and features for engaging uneven, hard (e.g., calcified), soft and non-circular shaped native valve tissue.

For example, in addition to those retaining features (e.g., annular flanges 150, toroidal annular flanges 250) described above, FIGS. 13A-13F show enlarged cross-sectional views of expandable prosthetic valve devices 300 having variations in the shape and configuration of the annular flanges and/or other retainers configured in accordance with additional embodiments of the present technology. For example, FIG. 13A shows a device 300 having a single annular flange 350 having a U-shaped dip 352 coupled to the support 110. In another embodiment, FIG. 13B shows a device 300 having a braided outer surface 112 of the support 110. Additionally, FIG. 13B shows the device 300 having an elongated retainer 440, similar to device 200 shown in FIGS. 12C-12D, that surrounds the outer surface 112 of the support 110 and extends beyond a length of the support 110 such that, upon deployment, the elongated sections of the elongated retainer 440 evert from proximal and distal ends 441 and 443 and roll in an inside-out fashion (in a roll direction opposite from the roll direction shown in FIGS. 12A-12B) to form toroidal annular flanges 450. FIGS. 13C and 13D show devices 300 having singular annular flanges 550 configured to engage the supra-annular surface (FIG. 13C) or the subannular surface (FIG. 13D), respectively. FIG. 13E shows a device 300 having a single annular flange 650 having upstream 652 and downstream 652 arms for engaging the supra-annular and subannular surfaces, respectively. FIG. 13F shows a device 300 having a plurality of looped flanges 750 along the outer surface 112 of the support 110. The devices 300 illustrated in FIGS. 13A-13F are only selected examples, and one of ordinary skill in the art will recognize that devices 100, 200, and 300 can be formed with multiple arrangements and configurations of retainers, flanges, fixation members, shapes, sizes, valve structures and other components associated with such devices.

Optionally, and in other embodiments, the valve structure 130, support 110 or the retainer 140 may be constructed to provide the elution or delivery of one or more beneficial drug(s) and/or other bioactive substances into the blood or the surrounding tissue. For example, the device 100 may be coated with various polymers to enhance its performance, fixation and/or biocompatibility. Additionally, the device 100 may incorporate cells and/or other biologic material to promote sealing, reduction of paravalvular leak or healing.

In any of the embodiments described herein, the device 100 may include an antiplatelet agent, including but not limited to aspirin, glycoprotein IIb/IIIa receptor inhibitors (including, abciximab, eptifibatide, tirofiban, lamifiban, fradafiban, cromafiban, toxifiban, XV454, lefradafiban, klerval, lotrafiban, orbofiban, and xemilofiban), dipyridamole, apo-dipyridamole, persantine, prostacyclin, ticlopidine, clopidogrel, cromafiban, cilostazol, and nitric oxide. In additional variations, the device 100 may include an anticoagulant such as heparin, low molecular weight heparin, hirudin, warfarin, bivalirudin, hirudin, argatroban, forskolin, ximelagatran, vapiprost, prostacyclin and prostacyclin analogues, dextran, synthetic antithrombin, Vasoflux, argatroban, efegatran, tick anticoagulant peptide, Ppack, HMG-CoA reductase inhibitors, and thromboxane A2 receptor inhibitors.

### Selected Systems and Methods for Delivery and Implantation of Artificial Heart Valve Devices

FIG. 8B shows the prosthetic valve device 100 in a delivery state 102 in which it can have a narrow overall profile in the collapsed configuration to be received through an inner lumen of a vascular catheter. To pass through an access site introducer, the delivery catheter diameter containing the collapsed prosthetic valve can be between 6 Fr. and 26 Fr. and, in some embodiments, between 10 Fr and 24 Fr, and in other embodiments between 20 Fr. and 26 Fr, in yet further embodiments, the delivery catheter diameter can be between 16 Fr and 24 Fr. In one embodiment the delivery catheter diameter can be 18 Fr, or in another embodiment, 24 Fr.

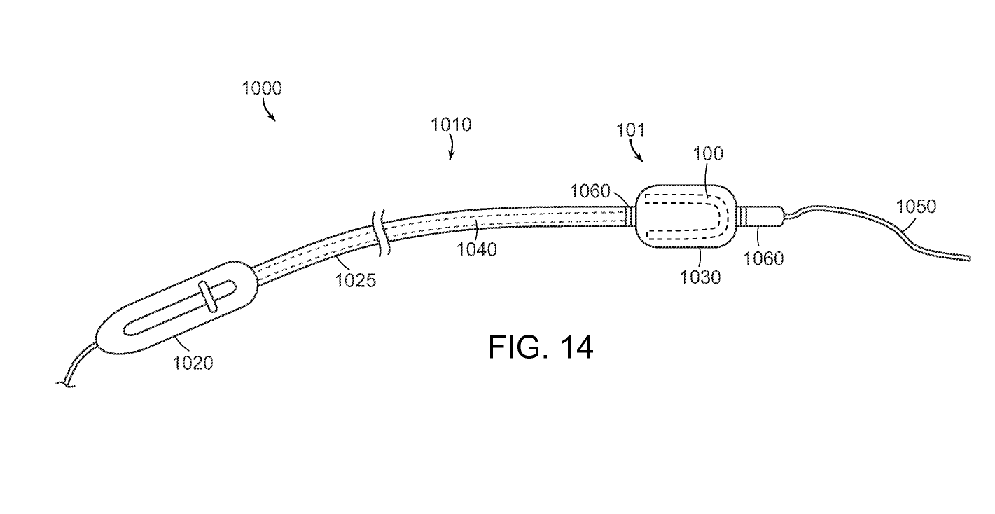

FIG. 14 illustrates a prosthetic valve device delivery system 1000 in accordance with an embodiment of the present technology. The system 1000 can include a prosthetic valve device 100, which can be any of the prosthetic valve device (e.g., device 100, 200 or 300) described herein, and a percutaneous heart valve delivery catheter 1010 configured to retain the device 100 in a delivery state 101 (e.g., collapsed configuration). In some embodiments, the delivery catheter 1010 may include a deployment handle 1020 with attached sheath 1025 and delivery sheath assembly 1030 containing the device 100 in a compressed arrangement over a catheter shaft 1040. The shaft may be hollow over all or any portion of its length to cooperate and follow a guidewire 1050. Actuating the deployment handle 1020 causes the sheath 1025 to be proximally retracted from the prosthetic valve device 100 and positioned in the valve annulus.

The precise positioning of the device 100 for native valve repair or replacement is important, particularly with respect to securing and maintaining the device 100 at the native annulus. Further, a device 100 that protrudes too far into the left atrium may cause a number of problems, including: disruption of atrial flow, reduction in atrial volume, high shear forces, promotion of thrombus formation, promotion of emboli formation, tissue erosion, etc. A device 100 that is positioned too far into the left ventricle may cause a number of problems, including: disruption of ventricle contraction, occlusion of the left ventricular outflow tract, promotion of thrombus formation, promotion of emboli formation, etc.

In some embodiments, radiopaque markers 1060 may be incorporated on the sheath 1035 and/or the shaft 1040 of the catheter 1010 at or otherwise flanking the delivery sheath assembly 1030 to assist in providing guidance on placement of the delivery sheath assembly 1030 before deployment of the device 100 (FIG. 14). Additionally, other radiopaque markers, not shown, may be incorporated into the annular flange 150 and/or the support 110 to help provide additional visibility under image guidance such as fluoroscopy, x-ray, and MRI. Marker materials may include: tungsten, tantalum, platinum, palladium, gold, iridium or other suitable materials.

Various methods known in the art for transcatheter delivery of devices, including artificial heart valve devices, can be used to deliver and employ the prosthetic valve devices described herein. Percutaneous delivery of devices to the mitral valve, or other atrioventricular valve can be accomplished by accessing the heart through a minimally invasive procedure of accessing a patient's vasculature through the skin in a location remote from the heart. Percutaneous access to remote vasculature is known in the art and several approaches to a target heart valve can be used using these techniques. For example, an approach to a mitral valve can be antegrade. An antegrade approach can include, for example, creating an endoluminal entry point in a femoral vein, iliac vein or right jugular vein of a patient. A guidewire may be introduced into the patient through the endoluminal entry point and advanced through the circulatory system, eventually arriving at the heart. Upon arriving at the heart, the guidewire is directed into the right atrium of the heart, traverses the right atrium via an atrial septum puncture, and enters the left atrium. The guidewire may then be advanced through the mitral valve while the heart is in diastole to the left ventricle.

Alternatively, approach to the mitral valve can be retrograde where the mitral valve may be accessed by an approach from the aortic arch, across the aortic valve, and into the left ventricle below the mitral valve with a guidewire. The aortic arch may be accessed a femoral artery access route, or via the brachial artery, axillary artery, or a radial or carotid artery. Use of the retrograde approach can eliminate the need for a trans-septal puncture.

A third approach to a mitral valve can include trans-apical puncture. In this approach, access to the heart is gained via thoracic incision, which can be a conventional open thoracotomy or sternotomy, or a smaller intercostal or sub-xyphoid incision or puncture. An access cannula is then placed through a puncture, sealed by a purse-string suture or other surgical technique, in the wall of the left ventricle near the apex of the heart. The catheters and prosthetic valve devices disclosed herein may then be introduced into the left ventricle through this access cannula.

Once percutaneous access is achieved, the interventional tools and supporting catheter (s) may be advanced to the heart intravascularly and positioned adjacent the target cardiac valve in a variety of manners, as described and known in the art. For example, once the guidewire is positioned, the endoluminal entry port is dilated to permit entry of a delivery catheter through the vasculature and along the guidewire path. In some instances, a protective sheath may be advanced in the venous area to protect the vascular structure.

After a guidewire is positioned by method briefly described above, an introducer can be advanced over the guidewire into the left atrium. A delivery catheter is inserted through the introducer. The valve is retained in a collapsed state in the distal end of the delivery catheter and advanced through the introducer. In some embodiments, the introducer may be formed with a tapered distal end portion to assist in navigation through the chordae tendineae or a flexible or removable dilator may used. The delivery catheter likewise can have a tapered distal end portion. The introducer can then be retracted relative to the delivery catheter to advance the valve assembly from the introducer, thereby allowing the entire assembly to expand to its functional size in an appropriate position for engagement of the device to the annulus. The introducer and catheter can then be withdrawn from the patient.

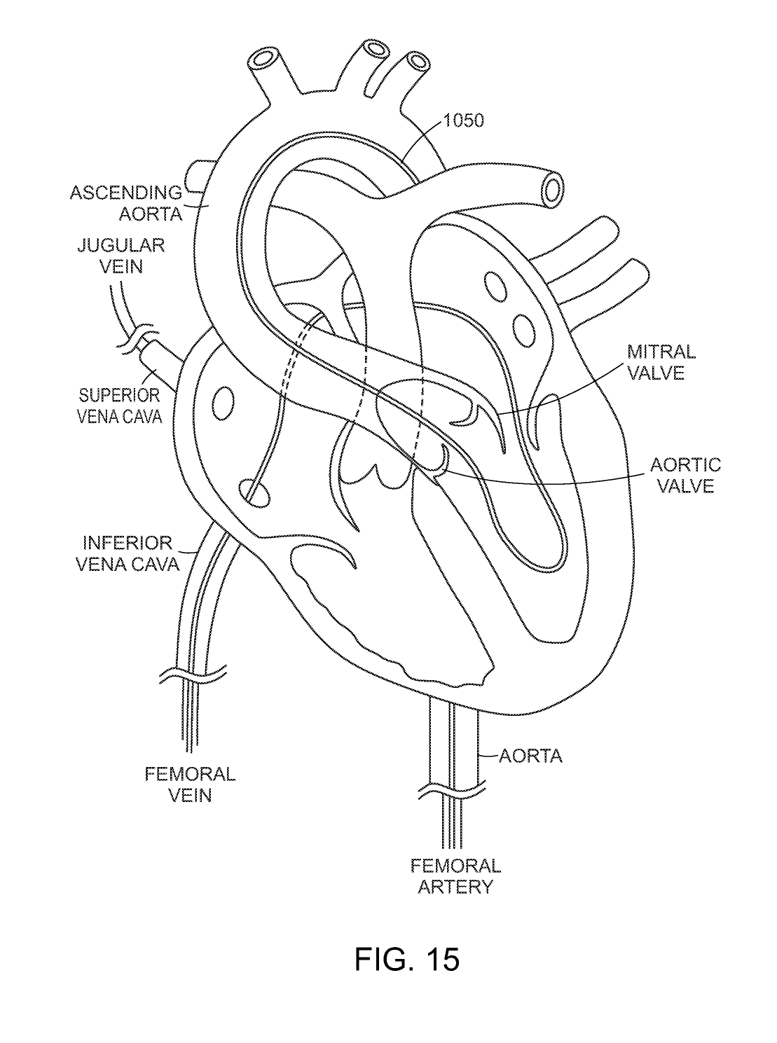

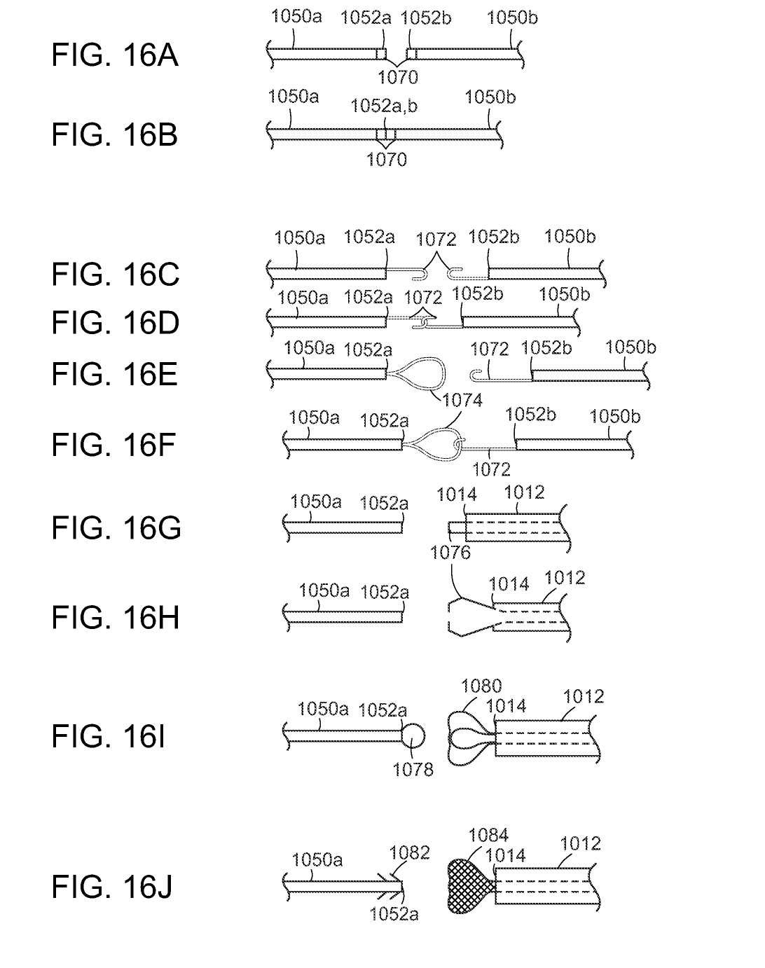

Additional methods for delivering a placing an expandable prosthetic valve device are further described below with respect to FIGS. 15-16J. FIG. 15 is a schematic illustration of a cross-sectional view of a heart showing a guidewire 1050 traveling along a guidewire path through the heart in accordance with an embodiment of the present technology. In one embodiment, a method for delivering and placing an expandable prosthetic valve device can include introducing a first guidewire through a first guidewire path. The first guidewire path can include passing the guidewire through the right femoral vein through to the inferior vena cava and into the right atrium. Optionally, the first guidewire can be introduced through the right jugular vein into the superior vena cava and into the right atrium. The guidewire can transverse the interatrial septum via a puncture and enter the left atrium. The first guidewire can then pass through the mitral valve into the left ventricle. The method can further include introducing a second guidewire through a second guidewire path different from the first guidewire path. The second guidewire path can include passing the guidewire through the femoral artery to the aorta and across the aortic valve into the left ventricle.

The first guidewire can have a first distal end and the second guidewire can have a second distal end, and the method can further include connecting the first distal end to the second distal end within the left ventricle (or other target chamber along the first or second guidewire paths) using an attachment mechanism coupled one or both of the first or second distal ends of the guidewires. Examples of attachment mechanisms can include a grasper, basket, snare, loop, hook, barb, magnet, brush, screw, corkscrew, latch, balloon or other suitable attachment components suitable in the art for connecting two separate ends of guidewires to each other. FIGS. 16A-16F illustrate embodiments of various attachment mechanisms suitable for coupling a first distal end 1052a of a first guidewire 1050a to a second distal end 1052b of a second guidewire 1050b, for example, within a target chamber of a heart. For example, FIGS. 16A and 16B show magnets 1070 located at each of the distal ends 1052a, 1052b that can be used to connect the distal ends 1052a, 1052b together to create a single guidewire 1052 spanning both of the first and second guidewire paths discussed above. In another embodiment, FIGS. 16C and 16D show hooks 1072 at each of the distal ends 1052a, 1052b. In a further embodiment, FIGS. 16E and 16F shown loop 1074 coupled to the first distal end 1052 of the first guidewire 1050a and the hook 1072 coupled to the second distal end 1052b of the second guidewire 1050b.

In some embodiments, after attaching the first and second guidewires using one or more attachment mechanisms, the first guidewire could be guided through the second guidewire path using a combination of actions such as pulling on the second guidewire and pushing the first guidewire so that a single guidewire traverses both the first and second guidewire paths. In another embodiment, the second guidewire could be pulled (and pushed) through the first guidewire path in a similar manner.

In another embodiment, one of the first or second guidewires can be exchanged for a catheter designed to couple to the remaining guidewire in the target chamber. For example, a catheter having an attachment mechanism on a distal end of the catheter can replace the second guidewire along the second guidewire path. The attachment mechanism at the distal end of the catheter can be used to couple the first distal end of the first guidewire and pull the first guidewire along the second guidewire path. FIGS. 16G-16J illustrate embodiments of various attachment mechanisms suitable for coupling the first distal end 1052a of the first guidewire 1050a to a catheter distal end 1014 of a catheter 1012, for example, within a target chamber of a heart. For example, FIGS. 16G and 16H show a grasper 1076 in a retracted position (FIG. 16G) and in a an advanced position (FIG. 16H) suitable for grasping or retaining the first distal end 1052a of the first guidewire 1050a. In another embodiment, FIG. 16I shows a snare cage 1080 at the catheter distal end 1014 suitable to snare a spherical member 1078 coupled to the first distal end 1052a of the first guidewire 1050a. In a further embodiment, FIG. 16J shows a braided member 1084 at the catheter distal end 1014 suitable to snare barbs 1082 or hooks formed at the first distal end 1052a of the first guidewire 1050a.

Once a single guidewire travels through both the first and second guidewire paths, a delivery catheter, such as delivery catheter 1010 shown in FIG. 14, housing a prosthetic valve device can be guided over the remaining guidewire to the native valve of interest (e.g., mitral valve, aortic valve). With a single guidewire traveling through the first and second paths, the delivery catheter can be positioned along the guidewire path using a combination of actions such as pulling on the guidewire so that the distal end of the delivery catheter is pulled into position at the native valve of interest along with pushing the catheter into place. In one embodiment, the catheter can be pulled and pushed through the aortic valve and turned within the left ventricle to approach the downstream or ventricular side of the mitral valve in an atraumatic manner (e.g., without unintentional damage to the aortic valve). Accordingly, the method described can also provide a physician or operator with improved control and placement of the valve assembly during delivery and deployment. Further, the method could enable femoral delivery of a prosthetic heart valve normally difficult to navigate along the second guidewire path. if the delivery catheter travels along the second guidewire path as described above, only the first guidewire need travel through the transeptal puncture. Accordingly, the diameter of the transeptal puncture between the left and right atrium could be reduced in such procedures.

Following delivery, placement and deployment of a prosthetic heart valve device at the desired valve location along the first or second guidewire paths, the delivery catheter and remaining guidewire can be removed from the heart and out of the body of the patient.

### Conclusion

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. An expandable prosthetic valve device for implantation at a native valve region of a heart, the device comprising:
a radially-expandable support having an expandable outer wall and a lumen defined by the outer wall;
a valve in the lumen and coupled to the support; and
a self-expanding retainer coupled to the outer wall of the support, the retainer including-
a structural braid configured to form a first annular flange on the outer wall of the support when the device is in a deployed configuration; and
an occlusive braid configured to reduce blood flow through the braid.

2. The device of claim 1, wherein the structural braid is configured to form a second annular flange, the second annular flange separated from the first annular flange by a gap, and wherein the gap is configured to receive an annulus at the native valve region.

3. The device of claim 2, wherein the first and second annular flanges provide a compressive force against the annulus.

4. The device of claim 1, wherein the occlusive braid is a first occlusive braid and wherein the self-expanding braid includes a second occlusive braid.

5. The device of claim 4, wherein the structural braid is between the first and second occlusive braids.

6. The device of claim 1, wherein the structural braid and the occlusive braid are interwoven.

7. The device of claim 1, wherein the structural braid is coupled to the support, and wherein the structural braid is between the support and the occlusive braid.

8. The device of claim 1, wherein the structural braid provides a radial force against the native valve region.

9. The device of claim 1, wherein the support has a central longitudinal axis and a first radial force in an outward, radial direction from the longitudinal axis, and wherein the retainer has a second radial force in an outward, radial direction from the longitudinal axis, and wherein the second radial force is less than the first radial force.

10-11. (canceled)

12. The device of claim 1, wherein the native valve region is a mitral valve annulus and wherein the annular flange is configured to engage the mitral valve annulus.

13. The device of claim 1, wherein the native valve region is an aortic valve annulus and wherein the annular flange is configured to engage the aortic valve annulus.

14-65. (canceled)

66. A method for delivering and placing an expandable prosthetic valve device, the method comprising:
introducing a first guidewire having a first distal end through a first path through a heart to a target chamber; and
introducing a second guidewire having a second distal end through a second path through the heart to the target chamber, the second path different than the first path.

67. The method of claim 66, wherein introducing a first guidewire having a first distal end through a first path includes-
passing the first guidewire from a right femoral vein to an inferior vena cava and into a right atrium;
puncturing a septum between the right atrium and a left atrium; and
passing the first guidewire across the septum into the left atrium and through a mitral valve to a left ventricle of the heart.

68. The method of claim 66, wherein introducing a second guidewire having a second distal end through a second path includes passing the second guidewire from a femoral artery to an aorta and through an aortic valve into the left ventricle.

69. The method of claim 66, wherein the target chamber is a left ventricle.

70. The method of claim 66 further comprising connecting the first distal end to the second distal end.

71. The method of claim 70, wherein at least one of the first distal end and the second distal end includes an attachment mechanism, and wherein connecting the first distal end to the second distal end further comprises coupling the first and second distal ends with the attachment mechanism.

72. (canceled)

73. The method of claim 66 further comprising pulling the first guidewire distally through the second path.

74. (canceled)

75. The method of claim 73 further comprising passing a delivery catheter housing the expandable prosthetic valve device over the first guidewire along the second path.

76. (canceled)

77. The method of claim 75, wherein the expandable prosthetic valve device is configured to replace a mitral valve, and wherein the delivery catheter places the expandable prosthetic valve device in the mitral valve of the heart.

78. (canceled)

79. The method of claim 66 further comprising pulling the second guidewire distally through the first path.

80. (canceled)

81. The method of claim 79 further comprising passing a delivery catheter housing the expandable prosthetic valve device over the second guidewire along the second path.

82. The method of claim 81, wherein the expandable prosthetic valve device is configured to replace an aortic valve, and wherein the delivery catheter places the expandable prosthetic valve device in the aortic valve of the heart.

83. The method of claim 81, wherein the expandable prosthetic valve device is configured to replace a mitral valve, and wherein the delivery catheter places the expandable prosthetic valve device in the mitral valve of the heart.

84-90. (canceled)
